(19) United States
(12) Patent Application Publication
(10) Pub. No.: US 2005/0065430 A1
(43) Pub. Date: Mar. 24, 2005

(54) Methods of cardiothoracic imaging - (MET-30)

(76) Inventors: Andrea Wiethoff, Cambridge, MA (US); Edward C. Parsons, JR., Burlington, MA (US); Rene Botnar, Chestnut Hill, MA (US); Elmar Spuentrup, Aachen (DE)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/888,833

(22) Filed: Jul. 9, 2004

Related U.S. Application Data
(60) Provisional application No. 60/486,833, filed on Jul. 10, 2003.
(60) Provisional application No. 60/543,875, filed on Feb. 12, 2004.

(51) Int. Cl. A61B 5/05

(52) U.S. Cl. 600/413; 600/420; 600/509

(57) ABSTRACT

Methods for imaging stationary targets, including thrombi, are disclosed. The methods allow the imaging of stationary targets in areas of the body subject to physiologic motion.

### CROSS-REFERENCE TO RELATED APPLICATIONS

This application claims the benefit of and priority under 35 U.S.C. § 119(e) to U.S. Provisional Applications Ser. Nos. 60/486,833, filed Jul. 10, 2003 and 60/543,875, filed Feb. 12, 2004, both of which are incorporated by reference in their entirety herein.

### TECHNICAL FIELD

This invention relates to magnetic resonance imaging, and more particularly to methods for imaging stationary targets, such as thrombi, in areas of the body subject to physiologic motion.

### BACKGROUND

Although MR imaging is a powerful diagnostic method for visualizing a variety of pathophysiologic and anatomic states at high resolution, a wide variety of artifacts are routinely encountered in MR images. One class of artifacts, motion artifacts, is inherent in the method itself in that MR imaging equations assume stationary objects. Object motion during the acquisition of MR image data produces both blurring and ghosting in the phase-encoded direction. One type of motion artifact, view-to-view motion effects, is caused by motion that occurs between the acquisition of successive phase-encoding steps, resulting in phase errors and ghosting in the MR images. Periodic physiologic motion due to the respiratory cycle, the cardiac cycle, vascular pulsation, and CSF pulsation result in such view-to-view motion effects. The other type of motion artifact results from motion occurring between the time of radiofrequency excitation and echo collection and is referred to as in-view motion. This type of motion typically changes the amplitude and phase of the MR signal as it evolves, resulting in blurring and increased noise in the image. In-view effects are most frequently associated with random motion, such as gastrointestinal peristalsis, swallowing, coughing, eye motion, and gross patient musculoskeletal motion. See, e.g., U.S. Pat. No. 6,184,682.

Stationary objects, such as thrombi or regions of infarcted myocardium within the cardiothoracic region, are particularly subject to motion artifacts resulting from musculoskeletal, cardiac, and respiratory motion. Even absent such motion, imaging of a thrombus or infarct remains difficult, often due to their relative size as compared to adjacent tissue (e.g., the heart) and the lack of sufficient contrast relative to background MR signal from flowing blood and adjacent fat and tissue. It would be useful to have methods for imaging stationary objects, such as thrombi and infarct, that would reduce motion artifacts in an MR image while nevertheless allowing sufficient contrast of the object in a reasonable imaging time frame.

### SUMMARY

The present invention is based on the finding that stationary objects, or stationary targets as referred to herein, in an animal's body can be successfully imaged despite their location in an area subject to physiologic motion. The present inventors have found that the combination of a targeted MR contrast agent and selective timing of MR data acquisition facilitates improved contrast and resolution of the stationary target.

Accordingly, in one embodiment, the invention provides a method for determining the presence or absence of a stationary target in a bodily location of an animal. An animal can be a mammal or a bird. A mammal can be a human, dog, cat, mouse, rat, pig, or monkey. The bodily location can be the heart, lung, kidneys, great blood vessels, or the liver. A bodily location can be the myocardium, an atrium, a ventricle, a coronary artery, or a valve of the heart. The bodily location can be subject to physiologic motion. The method includes:
a) administering a MRI contrast agent to said animal, with the MRI contrast agent capable of binding to the stationary target;
b) allowing the MRI contrast agent to bind to the stationary target; and
c) acquiring one or more MR images of the bodily location, wherein the acquisition of the one or more MR images is capable of reducing motion artifacts in the one or more MR images.

Physiologic motion can include periodic or nonperiodic (e.g., random) motion, or both. Periodic motion can be due to respiratory motion or cardiac motion of an animal. Nonperiodic motion can be due to musculoskeletal motion.

The reduction of motion artifacts can be achieved by acquiring MR data at a predetermined time during an animal's cardiac or respiratory cycle. In certain cases, MR data acquisition at a predetermined time during an animal's cardiac cycle occurs by coordinating MR data acquisition with a physiologic electrical or pressure signal of the animal. The physiologic electrical or pressure signal can be, for example, an ECG signal, a heartbeat, or a pulse. A pressure signal can be detected using an acoustic technique, an ultrasound technique, or a transducer. In other cases, a physiologic signal can be an ECG signal. MR data acquisition can occur during mid- or late-diastole of the ECG signal.

Acquisition of MR data during a predetermined period of an animal's respiratory cycle can occur by coordinating MR data acquisition with a location of an animal's diaphragm, liver, or lung. In certain embodiments, the location of a diaphragm, liver, or lung can be determined using a MR navigator, a tracking MR navigator, high speed MR projection images, or full MR images. In other cases, a predetermined period of a respiratory cycle can be determined by using a respiratory bellows. A predetermined period of an animal's respiratory cycle can be the beginning or end of expiration, or a breath-hold.

In certain embodiments, the one or more MR images can be acquired using a contrast-enhancing imaging pulse sequence. A contrast-enhancing imaging pulse sequence can be capable of suppressing the MR signal of in-flowing blood and can be further capable of enhancing the MR signal of the stationary target. A contrast-enhancing imaging pulse sequence can include a turbo field echo sequence, a spoiled gradient echo sequence, or a high speed 3D acquisition sequence. In certain cases, a contrast-enhancing imaging pulse sequence includes a black blood MR angiography sequence. A black blood MR angiography sequence can include a fast spin echo sequence, a flow-spoiled gradient echo sequence, an inversion recovery sequence, a double inversion recovery sequence, a fast gradient echo sequence, or an out-of-volume in-flow suppression sequence.

A contrast-enhancing imaging pulse sequence can include an in-flow-independent technique, which can be capable of enhancing the contrast ratio of a magnetic resonance signal of the stationary target having the MRI contrast agent bound thereto relative to a magnetic resonance signal of background blood or tissue. The background blood can be in-flowing blood. The background tissue can be fat, muscle, or tissue. An in-flow-independent technique can include an inversion-recovery prepared sequence, a saturation-recovery prepared sequence, a T₂ preparation sequence, or a magnetization transfer preparation sequence.

A stationary target can include a protein, such as fibrin, collagen, elastin, decorin, or a Toll-like receptor. In other cases, a stationary target is selected from the group consisting of oxidized LDL, matrix metalloproteinases, LTB4, and hyaluronan. A stationary target can be selected from the group consisting of a thromboembolism, an aneurism, an embolism, a thrombus, a tumor, a region of fibrosis, a region of infarcted tissue, a region of ischemic tissue, an atherosclerotic plaque, and a vulnerable plaque. A stationary target can be a region of heart, liver, kidney, or lung tissue, which may be ischemic or infarcted.

A contrast agent can be any contrast agent capable of binding to a stationary target or a component of a stationary target. In certain circumstances, a contrast agent can be selected from the group consisting of:

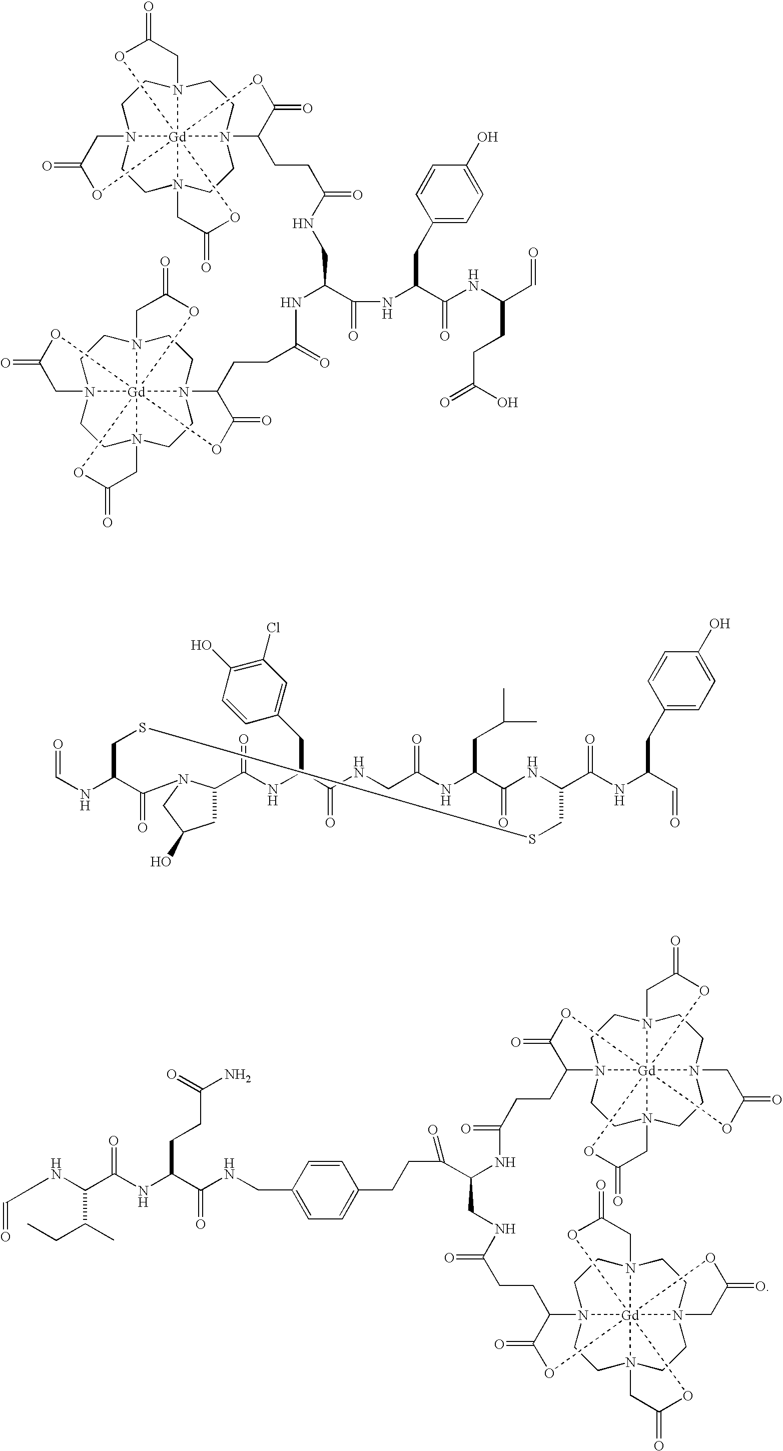

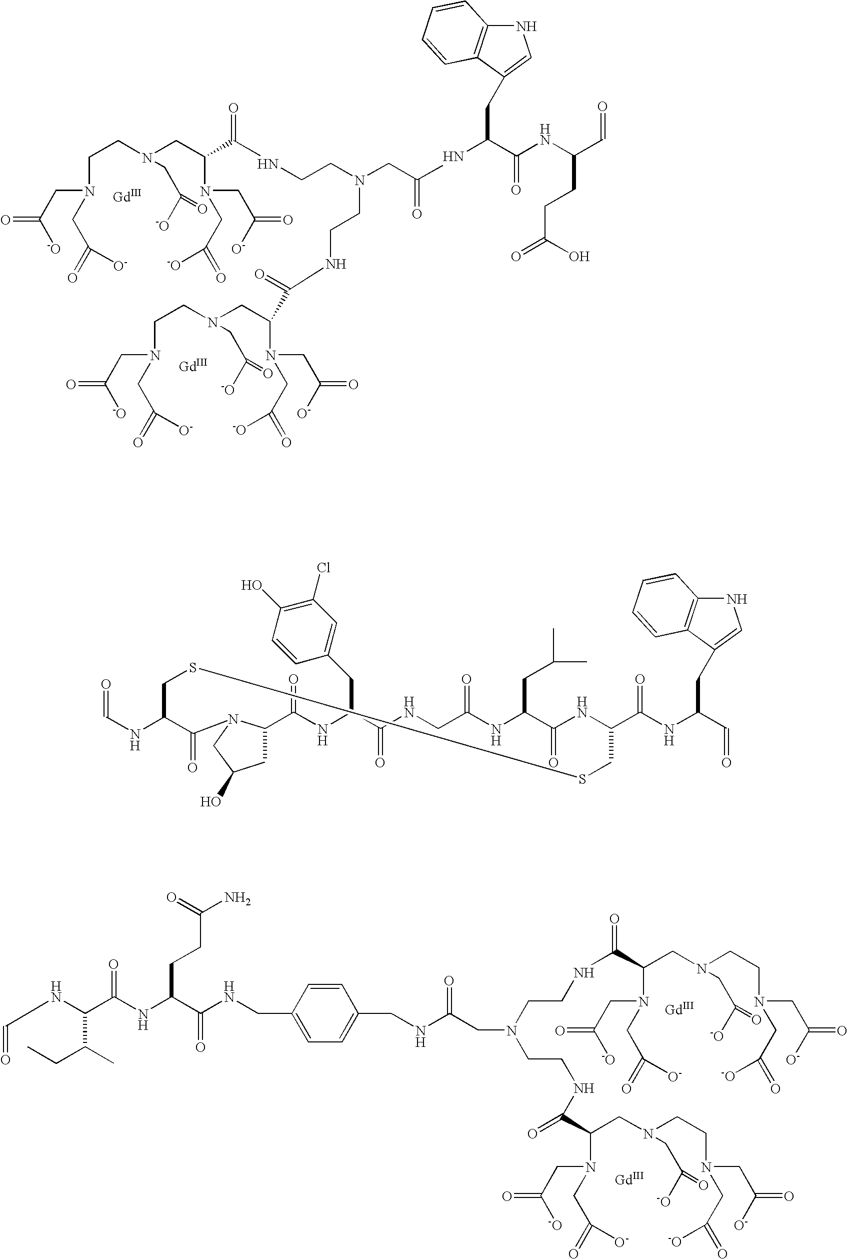

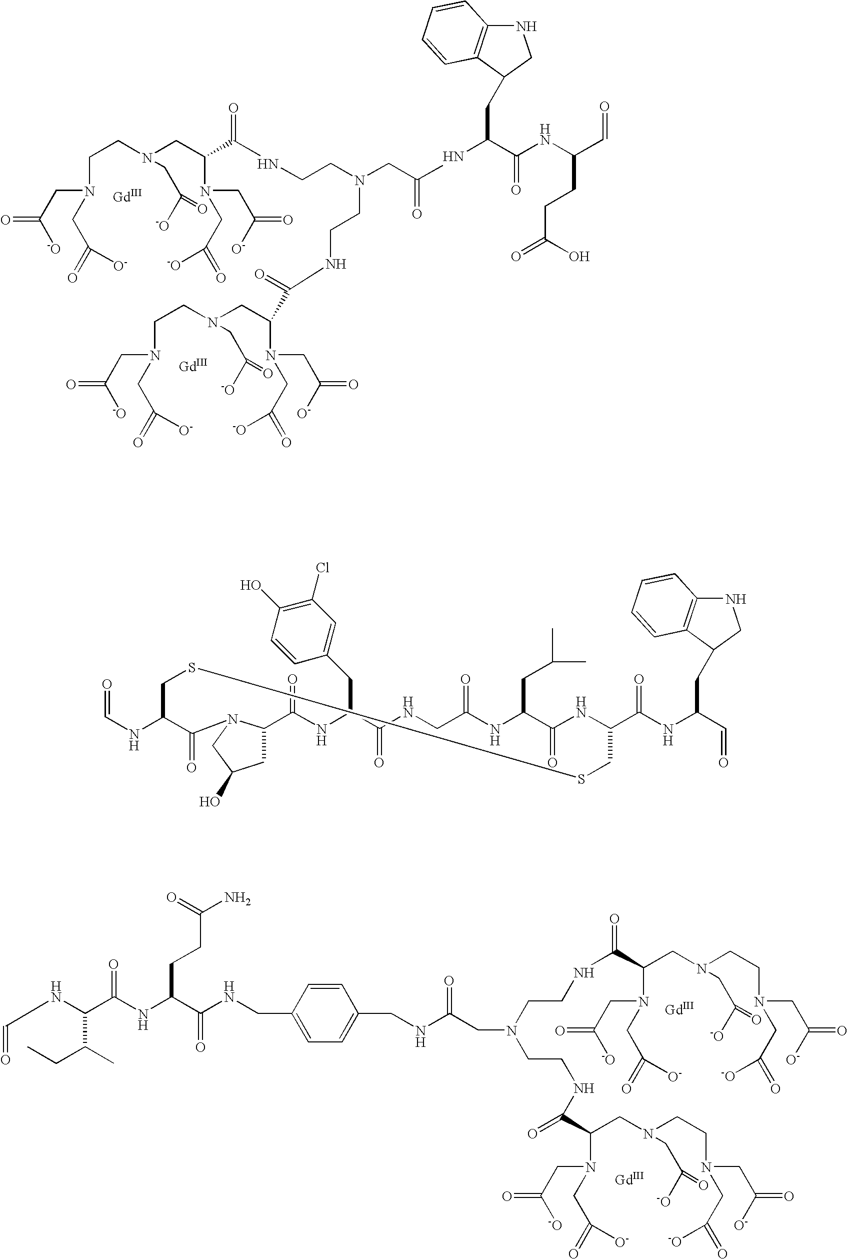

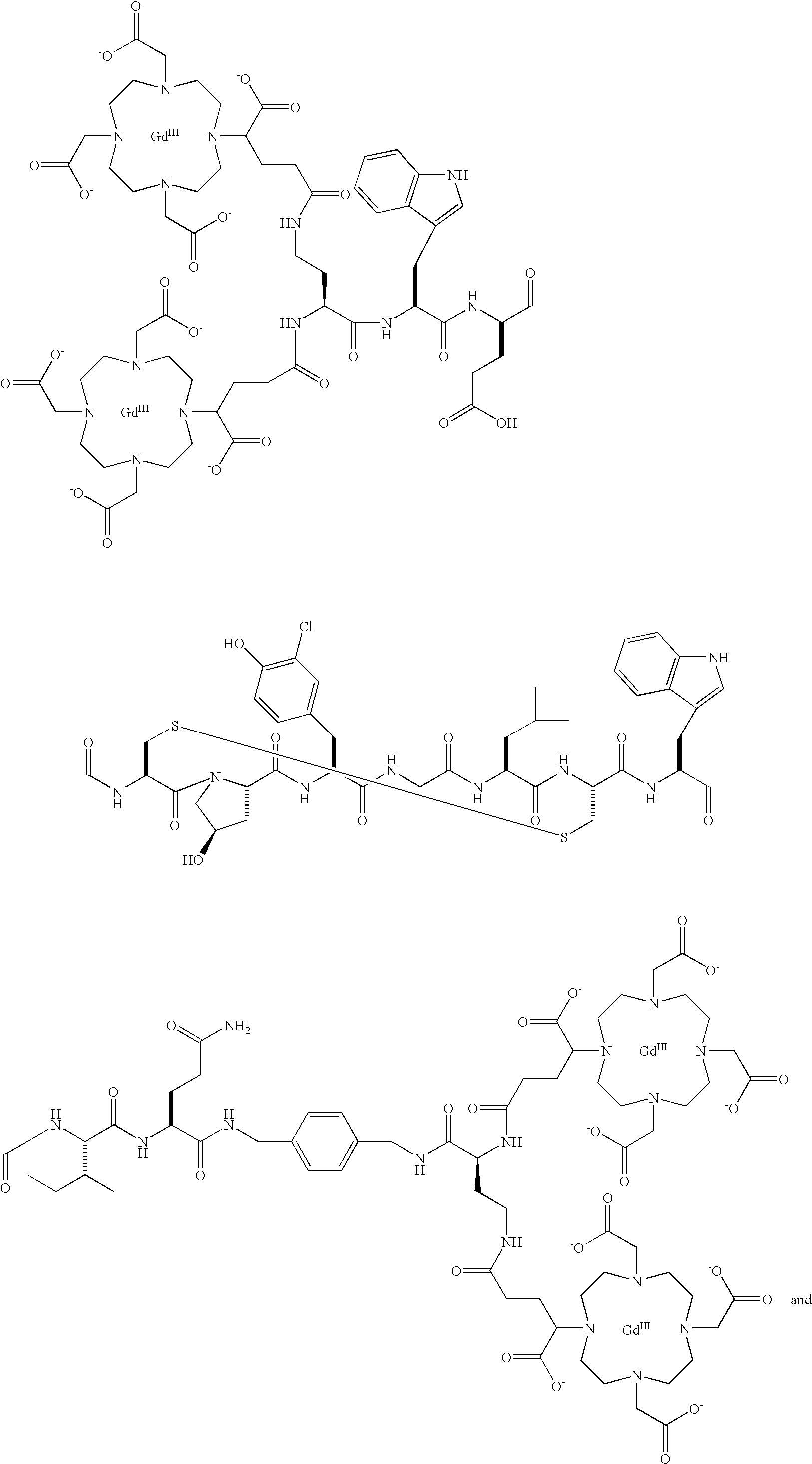

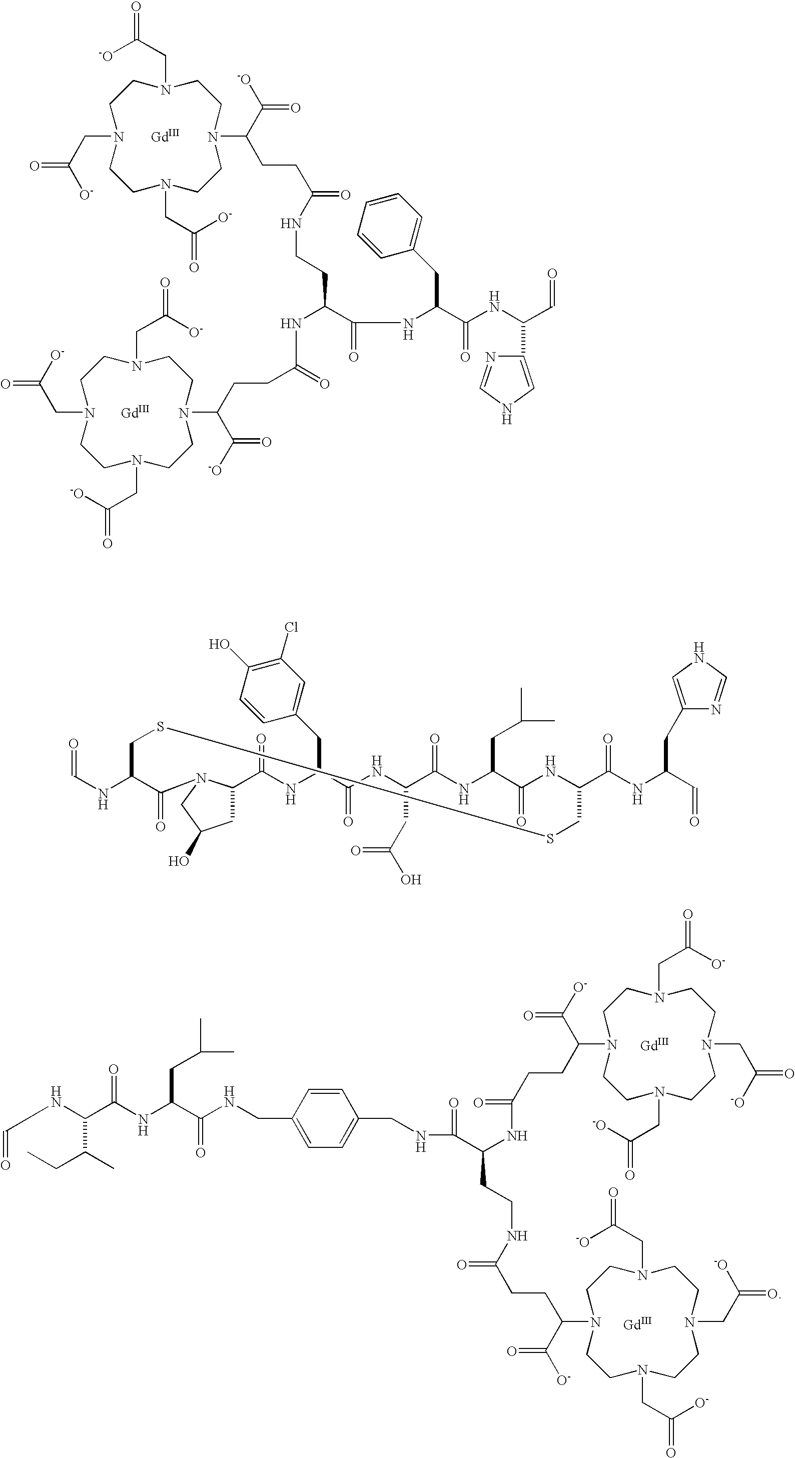

In another embodiment, the invention provides a method for determining the presence or absence of a stationary target in a bodily location of an animal, where the bodily location is subject to physiologic motion. The method includes:
a) administering a MRI contrast agent to the animal, the MRI contrast agent capable of binding to the stationary target;
b) allowing the MRI contrast agent to bind to the stationary target;
c) acquiring one or more MR images of the bodily location , where the acquisition of the one or more MR images is capable of reducing motion artifacts in the one or more NM images; and
d) examining the one or more MR images, where the stationary target is determined to be present when a contrast-enhanced region is observed. The presence of the contrast-enhanced region or stationary target can be correlated with a pathology of the animal. The pathology can be, for example, a coronary syndrome, a coronary stent thrombosis, fibrosis of the lung, ischemic myocardial tissue, infarcted myocardial tissue, a pulmonary embolism, and a deep venous thrombosis (e.g., DVTS).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 demonstrates schematics of ECG-triggered and navigator (NAV)-gated free-breathing MR pulse sequences: (A) bright blood balanced TFE (bTFE); (B) black blood inversion recovery (IR) TFE pulse sequence; (C) bright blood balanced TFE (bTFE); and (D) black blood inversion recovery (IR) TFE pulse sequence. Image acquisition was performed in mid-diastole, a quiescent period within the cardiac cycle. A frequency selective inversion prepulse (FatSat) was used for epicardial fat suppression (A, B, C, D). The IR-TFE sequences were preceded by a non-selective inversion pulse (B, D) with the inversion time set to null blood signal during data acquisition. A navigator restore pulse (B, D) was used to facilitate navigator gating.

FIG. 2 is a water phantom filled with both a clot prepared from native fibrinogen and a Gd-DTPA-labeled fibrin clot. A) Black blood IR-TFE image of Gd-labeled clot revealed excellent clot visualization for the Gd-labeled clot with high CNR and SNR (CNR<550; SNR<600). The native clot was difficult to delineate and had low CNR and SNR (CNR<8 SNR<18). B) Bright blood bTFE images showed a well delineated hypo-intense native clot and a slightly hyper-intense Gd-labeled clot with intermediate CNR and SNR (CNR<60; SNR<35 vs. CNR<23; SNR<112).

FIG. 3 demonstrates in vivo MR imaging of Gd-labeled fibrin clots. Views A) and D) demonstrate images acquired using coronary MRA sequences before and after clot delivery, respectively. On both scans, no apparent clot is visible (circle). Views B) and E) demonstrate images acquired using black blood inversion recovery TFE sequences before and after clot delivery, respectively. On the post clot delivery view (E), three bright areas are readily visible (arrows and circle), consistent with the location of clot delivery. No apparent clot was visible on the pre-clot image (B; arrow and circle). View C) demonstrate a late enhancement scan showing a hyper-enhanced (e.g., infarct or scar) septal wall, consistent with a LAD thrombus. View F) shows an X-ray angiogram confirming the MR finding of thrombus in the mid-LAD (circle). To allow comparison with MR images, the orientation of the X-ray image is horizontally reversed. LAD: left anterior descending; LCX: left circumflex.

FIG. 4 demonstrates in vivo MR imaging of coronary stent thrombosis. Bright blood bTFE images before (A) and after (D) stent placement and before (A) and after (D) injection of a fibrin binding MR contrast agent. No apparent thrombus and no stent artifacts are visible on the post stent placement and post contrast agent image (D). Black blood IR-TFE images before (B) and after stent placement (E). A bright spot suggestive of stent thrombosis is visible after intra-coronary injection of contrast agent and was subsequently confirmed by x-ray angiography (C, F). To allow comparison with MR images, the orientation of the X-ray images are horizontally reversed. LAD: left anterior descending. LCX: left circumflex.

FIG. 5 demonstrates in vivo MR imaging of coronary stent thrombosis. A) Black blood image post stent placement and post fibrin binding MR contrast agent administration reveals two thrombi in the mid-LCX (arrows). B) X-ray coronary angiogram confirming the MR findings. To allow comparison with MR images, the orientation of the X-ray image is horizontally reversed. LCX: left circumflex.

FIG. 6 demonstrates in vivo MR imaging of coronary thrombosis after systemic injection of a fibrin binding MR contrast agent. Bright blood bTFE (A) and black blood IR-TFE images before (C) and after (D) systemic injection of a fibrin-binding MR contrast agent. Good thrombus depiction (arrow) is evident in the post-contrast image (D). The thrombus was subsequently confirmed (arrow) by x-ray angiography (B); to allow comparison with M images, the orientation of the X-ray image is horizontally reversed.

FIG. 7 demonstrates in vivo MR imaging of pulmonary embolism before and after systemic injection of a fibrin binding MR contrast agent. (A): pre-contrast black blood gradient echo images; (B) post-contrast black blood gradient echo images. Good pulmonary embolism depiction (arrows). is evident in the post-contrast images. X-ray angiography confirmed the MR findings.

FIG. 8 demonstrates in vivo MR imaging of pulmonary embolism and coronary thrombosis before and after systemic injection of a fibrin binding MR contrast agent. (A): pre-contrast black blood gradient echo images; (B) post-contrast black blood gradient echo images. Good pulmonary embolism and coronary thrombosis depiction (arrows) is evident in the post-contrast images. X-ray angiography confirmed the MR findings.

### DETAILED DESCRIPTION

Definitions

The term in-flowing blood, as used herein, refers to blood which flows into a voxel, viewing area of interest, imaging volume, or imaging slab during data acquisition.

The term “relaxivity” as used herein, refers to the increase in either of the MRI quantities 1/T1 or 1/T2 per millimolar (mM) concentration of paramagnetic ion or contrast agent, wherein T1 is the longitudinal or spin-lattice, relaxation time, and T2 is the transverse or spin-spin relaxation time of water protons or other imaging or spectroscopic nuclei, including protons found in molecules other than water. Relaxivity is expressed in units of mM⁻¹ s⁻¹.

The terms “target binding” and “binding” for purposes herein refer to non-covalent interactions of a contrast agent with a target. These non-covalent interactions are independent from one another and may be, inter alia, hydrophobic, hydrophilic, dipole-dipole, pi-stacking, hydrogen bonding, electrostatic associations, or Lewis acid-base interactions.

As used herein, “stationary target” means a nonflowing tissue or region of bodily tissue. For example, a thrombus localized in a blood vessel would be considered nonflowing and therefore a stationary target. Flowing blood, on the other hand, would not be considered a stationary target.

As used herein, all references to “Gd,” “gado,” or “gadolinium” mean the Gd(III) paramagnetic metal ion.

This invention relates to MRI-based methods useful for imaging stationary targets in bodily locations subject to physiologic motion. Use of the methods can improve the quality of MR images of stationary targets, facilitating more accurate diagnosis of pathologies related to the presence of such stationary targets. Accordingly, the invention facilitates the differentiation between necrotic (acutely infarcted myocardium), ischemic, and viable myocardial tissue; the determination of the presence and size of coronary syndromes, including acute coronary syndromes (e.g., thrombi, thromboembolisms, embolisms, aneurisms, clots and atherosclerotic plaque, including vulnerable plaque; “red” or blood-rich thrombus associated with ST-elevation MI; and “white” fibrin and platelet rich thrombus associated with non-ST-segment elevation MI and unstable angina); the evaluation of fibrosis in the lungs; the localization and identification of lesions in the vasculature; and the diagnosis and localization of deep vein thrombosis.

A method described herein may facilitate the diagnosis of in-stent thrombosis and thrombi that result from placement of stents in the vasculature. Acute or subacute coronary thrombosis is a serious complication of coronary artery stenting. In recent outcome studies of elective angioplasty using modern stenting techniques and anti-platelet therapies (Gp IIb/IIIa), the incidence rate of coronary stent thrombosis was ˜1% with a median occurrence time of ˜1 day after stent placement. In patients with unstable angina, the incidence rate increased to ˜2-4%. Direct imaging of thrombosis therefore may be beneficial for both diagnoses and guidance of therapy in these patients.

Stationary Targets

Methods provided herein can allow the determination of the presence or absence of a stationary target in a bodily location subject to physiologic motion. Physiologic motion affecting MR resolution can include periodic or non-periodic (e.g., random) motion, or combinations of the two. Periodic motion can include, for example, respiratory motion, cardiac motion (e.g., the beating heart), vascular pulsation, or CSF pulsation. Nonperiodic motion, or random motion, can include, without limitation, musculoskeletal motion, peristalsis, swallowing, coughing, and eye motion. The methods of the present invention thus allow the reduction of motion artifacts affecting the imaging of stationary targets with contrast agents.

Typical bodily locations where methods of the present invention may be employed include the heart, the lungs, the kidneys, the great vessels (right and left brachiocephalic veins, left common carotid artery, right brachiocephalic artery, and left subclavian artery), and the liver. Within the heart, the myocardium, atria, ventricles, coronary arteries, and valves are also examples of bodily locations subject to physiologic motion. Skeletal joints are also bodily locations subject to physiologic motion and resultant motion artifacts in MR images.

Stationary targets can include thromboembolisms, aneurisms, embolisms, tumors, thrombi, fibrotic regions, atherosclerotic plaques (including vulnerable plaques), and tissue or regions in the heart, lungs, or liver, including regions that are ischemic or infarcted. A stationary target can result from an acute coronary syndrome (ACS), e.g., coronary plaque rupture with subsequent thrombosis, including “white” and/or “red” thrombus. A stationary target can include one or more proteins or extracellular matrix components, and the contrast agent employed in the method can exhibit affinity for the proteins or extracellular matrix components. Such an affinity can allow the contrast agent to bind to the stationary target. In such a case, the contrast agent is said to be “targeted” to the protein or extracellular matrix component of the stationary target.

One example of a useful protein in a stationary target is fibrin, found at high concentration in thrombi, clots, and plaques. Other useful targets include extracellular matrix components (e.g., of the myocardium), which can include soluble and insoluble proteins, polysaccharides, such as heteropolysaccharides and polysaccharides covalently bound to proteins, and cell-surface receptors. Typical examples include collagens (Types I, III, IV, V, and VI), elastin, decorin, glycosoaminoglycans, and proteoglycans. Glycosaminoglycans include hyaluronan (also called hyaluronic acid), dermatan sulfate, chondroitin sulfate, heparin, heparan sulfate, and keratan sulfate. Hyaluronan (HA) is a highly charged polyanionic glycosaminoglycan which is an abundant component of atherosclerotic lesions in humans, and has been implicated in a wide variety of other pathological processes, including wound healing, tumor invasion, and inflammation. Other extracellular matrix components include biglycan and versican.

Collagens are particularly useful extracellular matrix components. Collagens I and III are the most abundant components of the extracellular matrix of myocardial tissue, representing over 90% of total myocardial collagen and about 5% of dry myocardial weight. The ratio of collagen I to collagen III in the myocardium is approximately 2:1, and their total concentration is approximately 100 μM in the extracellular matrix.

Another useful extracellular matrix component to target is elastin. The aorta and major blood vessels are 30% by dry weight elastin. Similarly, proteoglycans are also suitable for targeting, including proteoglycans present in the heart and blood vessels. For example, in non-human primates, proteoglycan distribution in the left ventricle is approximately 62% heparan sulfates; 20% hyaluronan, and 16% chondroitan/dermatan sulfates. The choindroitan/dermatan sulfate fraction consists exclusively of biglycan and decorin. Finally, Toll-like receptors, matrix metalloproteinases, oxidized LDL, and leukotrienes can also be targeted by the contrast agent. Toll-like receptors (TLR) are involved in immune reactions against bacteria. In addition to their role in immune response, TLRs have recently been investigated for their role in atherosclerosis. In apoE-deficient mice that were fed a high-fat diet, for example, TLR-4 was expressed in aortic root lesions, while no expression was seen in the aortic tissue of control mice.

Triggering and Gating Techniques

In general, a method provided herein can provide contrast-enhanced MR imaging of a moving bodily region (e.g., the cardiothoracic region) and stationary targets (e.g., thrombi) within such a moving bodily region. A method can include administering an MR contrast agent to an animal. An animal can be a mammal, such as a human, dog, pig, cat, monkey, mouse, or rat, or a bird. MR contrast agents can be capable of binding to a stationary target or a component of a stationary target, as described above. One or more MR images of a bodily location, e.g., a bodily location suspected to have all or part of a stationary target located therein, can be acquired. The acquisition can be capable of reducing motion artifacts in the MR images. For example, reduction of motion artifacts can be achieved by acquiring MR data at a predetermined time in an animal's cardiac cycle, or during a predetermined period of an animal's respiratory cycle. Thus, data acquisition can be timed in order to localize sampling of data in time such that the image reflects the bodily region, e.g., the heart, in a given static position.

Two classes of techniques are generally used to localize the MR image of the heart to reduce motion artifacts. One technique is a prospective triggering to the cardiac cycle. Cycles of MR data acquisition are commenced at (e.g., coordinated with) a predetermined or particular point in the cardiac phase, such as mid to late-diastole when heart motion is usually at a minimum. A cardiac phase or cycle may be detected using electrical or pressure signals of an animal (e.g., tracking of ECG signals, heartbeats, or the pulse of an animal). Pressure signals may be monitored using acoustic or ultrasound techniques and transducers.

The second technique, called gating, tracks respiratory motion. Gating can be done prospectively or retrospectively. The respiratory cycle or phase can be monitored with electophysiological or pressure measurements, by MR monitoring (e.g., high speed MR projection images, full MR images), or by using MRI navigation. MRI navigators are methods of quickly acquiring low-resolution pilot images taken once per image cycle (generally a heartbeat), usually to indicate the relative position of the lungs, liver, and/or diaphragm. For example, projections of the dome of the right hemi-diaphragrn can be acquired, where motion is most exaggerated and the junction of liver tissue and air in the lung provides demarcation. The navigator image is generally acquired at a location in the periphery of the imaging volume, at a line defined by the intersection of two planes defined by oblique gradients and slice-selective RF pulses. The navigator acquisition is interspersed with the image acquisition cycle, so as not to interfere with the spatial and temporal location of the primary image acquisition.

The position of, for example, the diaphragm is assessed with each navigator in real-time, using an edge detection algorithm. If the edge falls within a pre-specified window, the primary image data acquired during that cycle is retained, and the next segment of image data is sampled in the next cycle. Otherwise, the data are not accepted, and the same segment of image data is re-acquired in the next cycle. The acceptance window can be expanded without sacrificing image quality if the deflection of the image volume is estimated from the diaphragm position. A certain degree of deflection can be corrected for, either by altering the position of the sampled image volume to follow the motion of the chest, or by manipulating the data after acquisition to shift it to the target position so that all data is spatially co-registered in the reconstructed image. This approach has been implemented to increase the time efficiency of the overall imaging process.

Accordingly, by using an MR navigator, MR data acquired during a predetermined phase of the respiratory cycle is achieved by tracking the position of the lungs, liver, and/or diaphragm and by accepting data acquired during a particular positioning of the lungs, liver, and/or diaphragm. Thus, the acquisition of MR data is coordinated to the location of an animal's diaphragm, liver, and/or lung. MR navigators for use in the present invention are known to those of skill in the art, and can include tracking MR navigators.

A predetermined period of a respiratory cycle may be the beginning or end of expiration, or may be a breath-hold. A navigator method can also be used to adjust the image acquisition position to follow the relative position of the chest. A respiratory bellows can be used to indicate the respiratory cycle.

Contrast-Enhancing Imaging Pulse Sequences

Methods of the present invention can include the use of contrast-enhancing imaging pulse sequences. Such sequences are generally known to those of skill in the art. The pulse sequences, can be chosen to allow sufficient contrast of the stationary target in a reasonable imaging time frame, given the effect of the combination of the navigator and/or triggering techniques, the affinity of the contrast agent for the target, the contrast agent's half-life, and the effects of in-flowing blood and enhancement of background tissue and/or fat on image resolution. The contrast modes available in MR imaging may be constrained in certain instances by the timing parameters monitored by triggering and gating. For example, the acquisition window may be constrained to the intersection of mid- to late-phase diastole and the end of expiration. Rapid imaging techniques can be used, or data from several acquisition windows can be concatenated in order to reconstruct images in 2 or 3 dimensions.

In the absence of physiologic motion, fast imaging pulse sequences are generally used to bring an animal to steady-state equilibrium by running “dummy scans” or “dummy pulses” for a short period before data acquisition. When imaging in the presence of physiologic motion, however, it may not be desirable to delay data acquisition for this period of time. In addition, in the presence of physiologic motion, issues of unsaturated in-flowing blood, circuitous and multi-directional blood flow, and the transient response of the blood to the imaging pulses may need to be addressed. For example, the blood signal can be nulled, e.g., globally nulled.

To null signal from blood, a non-selective inversion recovery (IR) prepulse can be used. This inverts the nuclear magnetization, which then returns to its positive equilibrium value with an exponential rate given by the T1 time. In blood, this time is about 1200 ms. Thus, the magnetization passes through zero at a time determined by the initial magnetization, or about 300 ms after the IR prepulse for cardiac triggering cycles in normal physiological range for humans. At that time, image acquisition can commence with negligible contamination from blood signal. This method can be implemented as a single global IR inversion pulse, or as a dual-IR pulse. Because one may want to image targets with T1 similar to that of blood that may also be nulled by the IR pulse, a second, slice selective pulse restores equilibrium to positive equilibrium within the image volume. Such a method allows visualization of stenoses in the coronary arteries, where the artery lumen is black, and myocardium and vessel wall are bright.

In seeking to image fibrin or clots in blood vessels, the myocardium and vessel walls can be suppressed relative to the targeted clot and the blood can be nulled to avoid obscuring the clot within the lumen. The distribution of a targeted contrast agent can be affected by binding parameters, diffusivity of the contrast agent, pharmacokinetic and timing parameters, and the composition of the clot. These parameters can be adjusted in certain circumstances to create a region of low-T1 water within the thrombus over a region of similar magnitude to an image voxel. Further, the pulse sequence timing can be engineered to maximize the thrombus signal relative to background myocardium, pericardium, and vessel wall, given the timing constraints of MRI in the presence of cardiac and respiratory motion. Further, IR pre-pulse timing can be tuned to diminish both background tissue and in-flowing blood.

Generally, the contrast-enhancing pulse sequence can be capable of suppressing the MR signal of in-flowing blood and also enhance the MR signal of the stationary target. Typical pulse sequences include a turbo field echo sequence, a spoiled gradient echo sequence, or a high speed 3D acquisition sequence.

A pulse sequence can include a black blood MR angiography sequence. Nonlimiting examples of such sequences include fast spin echo sequences, flow-spoiled gradient echo sequences, inversion recovery sequences, double inversion recovery sequences, fast gradient echo sequences, and out-of-volume in-flow suppression sequences.

A contrast-enhancing imaging pulse sequence can include an in-flow independent technique that is capable of enhancing a contrast ratio of a magnetic resonance signal of the stationary target having the MRI contrast agent bound thereto relative to a contrast ratio of a magnetic resonance signal of background blood or tissue. Background blood and tissue include in-flowing blood, fat, muscle, or tissue parenchyma. Nonlimiting examples of such in-flow independent techniques include inversion-recovery prepared sequences, saturation-recovery prepared sequences, T2 preparation sequences, or magnetization transfer (MT) preparation sequences. Inversion preparation, T2 preparation, or MT preparation may be implemented between the triggering event and the data acquisition window to increase T1, T2, and MT contrast. To limit signal from blood in the vasculature, in-flow suppression can be implemented via saturation recovery or an inversion recovery (IR) prepulse. The latter can be implemented as a single, non-selective IR pulse to null blood signal globally, or as a dual-IR pulse where the second, slice selective pulse restores equilibrium to better image long T1 features in-slice.

Contrast Agents

A contrast agent for use in the present invention can target the stationary target (or a component thereof, including a proteinaceous or extracellular matrix component of the stationary target) and bind to it, allowing MR imaging of the stationary target. Contrast agents of the invention can be any contrast agent capable of binding to the stationary target. In certain embodiments, at least 10% (e.g., at least 50%, 80%, 90%, 92%, 94%, or 96%) of the contrast agent can be bound to the desired target at physiologically relevant concentrations of contrast agent and target. The extent of binding of a contrast agent to a target can be assessed by a variety of methods known to those having ordinary skill in the art, e.g., equilibrium binding methods such as ultrafiltration. For measuring binding to a lesion or plaque, a blood vessel containing a lesion or plaque may be isolated and contacted with a contrast agent. After an incubation time sufficient to establish equilibrium, the solution of contrast agent in the blood vessel is removed, e.g., by aspiration. The concentration of unbound agent in the solution so removed is then measured. In both methodologies, the concentration of bound contrast agent is determined as the difference between the total concentration initially present and the unbound concentration following the binding assay. The bound fraction is the concentration of bound agent divided by the concentration of total agent.

Contrast agents can exhibit high relaxivity as a result of target binding (e.g., to fibrin in a thrombus), which can lead to better image resolution. The increase in relaxivity upon binding is typically 1.5-fold or more (e.g., at least a 2, 3, 4, 5, 6, 7, 8, 9, or 10 fold increase in relaxivity). Targeted contrast agents having 7-8 fold, 9-10 fold, or even greater than 10 fold increases in relaxivity are particularly useful. Typically, relaxivity is measured using an NMR spectrometer. The preferred relaxivity of an MRI contrast agent at 20 MHz and 37° C. is at least 10 mM-1s-1 per paramagnetic metal ion (e.g., at least 15, 20, 25, 30, 35, 40, or 60 mM-1s-1 per paramagnetic metal ion). Contrast agents having a relaxivity greater than 60 mM-1s-1 at 20 MHz and 37° C. are particularly useful.

Targeted contrast agents can also be taken up selectively by stationary targets such as clots, thrombi, plaques (e.g., atherosclerotic and vulnerable plaque), aneurisms, embolisms, tumors, fibrotic regions, infarts, ischemic tissues and regions, and lesions. Selectivity of uptake can be determined by comparing the uptake of the agent by the target as compared to the uptake by blood. The selectivity of contrast agents also can be demonstrated using MRI and observing enhancement of stationary target signal as compared to blood signal.

Typically, the contrast agent will have an affinity for the stationary target. For example, if the stationary target includes a protein, the contrast agent can bind the protein with a dissociation constant of less than 10 μM (e.g., less than 10 μM, less than 5 μM, less than 1 μM, or less than 100 nM).

Generally, the contrast agent can include one or more physiologically compatible chelating ligands (C) and one or more targeting groups (TG). A contrast agent can include one or more optional linkers (L), e.g., to connect a TG to a C. The contrast agent can include a targeting group that exhibits affinity for any component, or more than one component, of the stationary target. The targeting group can include a small organic molecule. The targeting group can include chromogenic or fluorogenic components, such as azo dyes or fluorophores. Peptides can be particularly useful for inclusion in a target group. For example, a peptide can be a point of attachment for one or more chelates at one or both peptide termini, optionally through a linker (L). A peptide can have from about 2 to about 75 amino acids (e.g., from about 3 to about 15 amino acids, from about 5 to about 13 amino acids, from about 9 to about 15 amino acids, or from about 10 to about 20 amino acids) and can be linear or cyclic. A peptide can include natural or non-natural amino acids, and can be capped at either or both termini. For example, a peptide can include a halogenated tyrosine (e.g., 3-fluoro, 3-chloro, 3-iodo, or 3-bromo tyrosine) or an hydroxyproline residue. In certain circumstances, a peptide can have the sequence shown in Example 1.

The C can be any of the many known in the art, and includes, for example, cyclic and acyclic organic chelating agents such as DTPA, DOTA, HP-DO3A, DOTAGA, and DTPA-BMA. The C can be complexed to a paramagnetic metal ion, including Gd(III), Fe(III), Mn(II), Mn(III), Cr(III), Cu(II), Dy(III), Ho(III), Er(III), Pr(III), Eu(II), Eu(III), Tb(III), and Tb(IV). Additional information regarding C groups and synthetic methodologies for incorporating them into the contrast agents of the present invention can be found in WO 01/09188, WO 01/08712, and U.S. patent application Ser. No. 10/209,183, entitled “Peptide-Based Multimeric Targeted Contrast Agents,” filed Jul. 30, 2002. In certain embodiments, the C DOTAGA may be preferred. The structure of DOTAGA, shown complexed with Gd(III), is as follows:

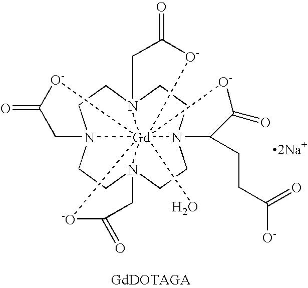

In other embodiments, the contrast agent can be an iron-based particle, e.g., as disclosed in U.S. Pat. Nos. 4,863,715; 4,795,698; 4,849,210; 4,101,435; 4,827,945; 4,770,183, and 5,262,176. In addition, the contrast agent can include one or more metal chelates bound to the surface of a particle, such as a gold, platinum, silver, or palladium particle or an inorganic particle made of silica, alumina, zirconia, calcium phosphate, or titania.

Contrast agents for use in the present invention are described in, for example, WO 03/011115 and WO 03/011113 and U.S. Pat. Nos. 6,676,929 and 6,652,835.

Pharmaceutical Compositions

Contrast agents used in the invention can be formulated as a pharmaceutical composition in accordance with routine procedures. As used herein, the contrast agents of the invention can include pharmaceutically acceptable derivatives thereof. “Pharmaceutically acceptable” means that the agent can be administered to an animal without unacceptable adverse effects. A “pharmaceutically acceptable derivative” means any pharmaceutically acceptable salt, ester, salt of an ester, or other derivative of a contrast agent of this invention that, upon administration to a recipient, is capable of providing (directly or indirectly) a contrast agent of this invention or an active metabolite or residue thereof. Other derivatives are those that increase the bioavailability of the contrast agents of this invention when such are administered to a mammal (e.g., by allowing an orally administered compound to be more readily absorbed into the blood) or which enhance delivery of the parent compound to a biological compartment (e.g., the brain or lymphatic system) thereby increasing the exposure relative to the parent species. Pharmaceutically acceptable salts of the contrast agents of this invention include counter ions derived from pharmaceutically acceptable inorganic and organic acids and bases known in the art, including sodium, calcium, and N-methyl-glucamine.

Pharmaceutical compositions of the invention can be administered by any route, including both oral and parenteral administration. Parenteral administration includes, but is not limited to, subcutaneous, intravenous, intraarterial, interstitial, intrathecal, and intracavity administration. When administration is intravenous, pharmaceutical compositions may be given as a bolus, as two or more doses separated in time, or as a constant or non-linear flow infusion. Thus, compositions of the invention can be formulated for any route of administration.

Typically, compositions for intravenous administration are solutions in sterile isotonic aqueous buffer. Where necessary, the composition may also include a solubilizing agent, a stabilizing agent, and a local anesthetic such as lidocaine to ease pain at the site of the injection. Generally, the ingredients will be supplied either separately, e.g. in a kit, or mixed together in a unit dosage form, for example, as a dry lyophilized powder or water free concentrate. The composition may be stored in a hermetically sealed container such as an ampule or sachette indicating the quantity of active agent in activity units. Where the composition is administered by infusion, it can be dispensed with an infusion bottle containing sterile pharmaceutical grade “water for injection,” saline, or other suitable intravenous fluids. Where the composition is to be administered by injection, an ampule of sterile water for injection or saline may be provided so that the ingredients may be mixed prior to administration. Pharmaceutical compositions of this invention comprise the contrast agents of the present invention and pharmaceutically acceptable salts thereof, with any pharmaceutically acceptable ingredient, excipient, carrier, adjuvant or vehicle.

A contrast agent is preferably administered to the patient in the form of an injectable composition. The method of administering a contrast agent is preferably parenterally, meaning intravenously, intra-arterially, intrathecally, interstitially or intracavitarilly. Pharmaceutical compositions of this invention can be administered to mammals including humans in a manner similar to other diagnostic or therapeutic agents. The dosage to be administered, and the mode of administration will depend on a variety of factors including age, weight, sex, condition of the patient and genetic factors, and will ultimately be decided by medical personnel subsequent to experimental determinations of varying dosage followed by imaging as described herein. In general, a dosage required for diagnostic sensitivity or therapeutic efficacy will range from about 0.001 to 50,000 μg/kg, preferably between 0.01 to 25.0 μg/kg of host body mass. The optimal dose may be determined empirically.

Methods

The methods of the invention allow the determination of the presence or absence of a stationary target in a bodily location subject to physiologic motion. Typically, an MRI contrast agent as described above is administered to the animal, the contrast agent is allowed to bind to the stationary target (if present), and one or more MR images of the bodily location are acquired. A contrast agent can be administered systemically, e.g., intravenously, as discussed previously, or through a stent. The images are acquired in a manner capable of reducing motion artifacts in the MR images. For example, the motion artifacts can be reduced by acquiring MR data at a predetermined time during the animal's cardiac or respiratory cycle, such as by triggering or gating MR data acquisition, as described above.

One or more acquired images can be examined for contrast-enhanced regions. A contrast-enhanced region can be indicative that a stationary target is present. A contrast-enhanced region or stationary target can also be correlated with a pathology of an animal, such as a coronary syndrome, a coronary stent thrombosis, fibrosis (e.g., of the lung), ischemic tissue (e.g., ischemic myocardial, liver, lung, or brain tissue), a pulmonary embolism, or a deep vein thrombosis.

In-Stent or Stent-Derived Thrombosis Imaging

Methods of the invention are also useful for imaging in-stent or stent-derived thrombi. Thrombi that result from the placement of stents, e.g., intracoronary stents, are a particular health concern. Typically, MR-lucent stents are used to prevent signal interference. A contrast agent as described previously is administered to the animal and allowed to bind to the stationary target, which will typically be a thrombus in or adjacent to a stent. One or more images of the bodily location containing the stent are then acquired. If the stent is in a bodily location subject to physiologic motion (e.g., the coronary arteries), the images may be acquired in a manner to reduce motion artifacts, as described previously. Contrast-enhancing imaging pulse sequences, as described above, may also be used in the method. See Example 1, below.

### EXAMPLES

### Example 1

### Coronary MR Angiography and Coronary Thrombus Imaging with Cardiac Triggering and Navigator Gating

Free-breathing coronary MR angiography and thrombus imaging were performed on six female domestic swine (70-80 kg) in the supine position using an interventional 1.5 T Philips Gyroscan ACS-NT short-bore MRI scanner. The MRI system was equipped with a specially shielded C-arm fluoroscopy unit (Philips Medical Systems, Best, NL), MASTER gradients (23 mT/m, 105 mT/m/ms), an advanced cardiac software patch (INCA2), and a 5-element cardiac synergy receiver coil.

Animal Protocol

After intramusculary premedication with 0.5 ml atropine and 0.2 ml azaperone/kg body weight, an aqueous solution of pentobarbital (1:3) was administered intravenously through one of the ear veins. The animals were intubated and mechanical ventilation was maintained throughout the entire experiment. A 9F sheath (Cordis, Roden, NL) was placed surgically in the right carotid artery.

MRI of Thrombi

The feasibility of direct coronary MR thrombus imaging was tested in vitro and in 3 animals after delivery of Gd-DTPA labeled fibrin clots (˜250μM Gd) to the left coronary artery (LCA) system. MR imaging of coronary stent thrombosis was investigated in another 3 animals after placement of novel MR-lucent stents and intra-coronary injection of a fibrin-binding MR contrast agent having the structure shown below and prepared as described in WO 03/011115: 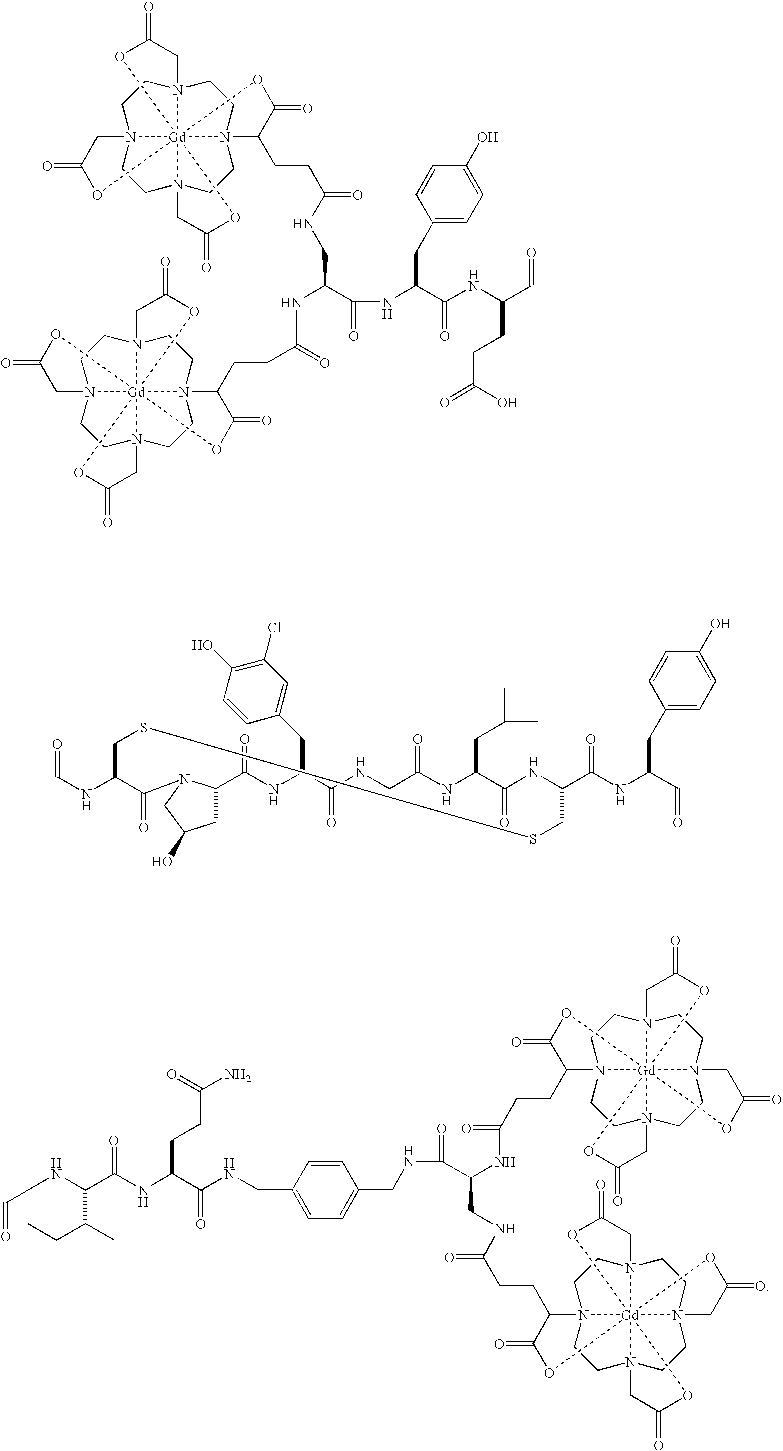
Imaging of Gd-DTPA Labeled Fibrin Clots

Four thrombi were engineered using Gd-DTPA labeled human fibrinogen (˜250 μM Gd), 10 NIH units thrombin (to cleave the fibrinogen to fibrin and result in clot formation), 25 mM CaCl_{2,} and fresh pig blood. Three Gd-DTPA fibrin clots were then delivered under x-ray guidance into the left coronary artery system of 3 female domestic swine using a 9F guiding catheter. These thrombi subsequently broke up into 4 LAD and 2 LCX clots. The remaining Gd-DTPA labeled clot was placed together with a native unlabeled fibrin clot in a water bath and served as an in vitro control. Free-breathing bright blood balanced Turbo Field Echo (bTFE; for coronary MR angiography imaging) and black blood IR-TFE (for MR thrombus imaging) 3D imaging of the LAD or LCX were performed before and after Gd-DTPA-loaded clot delivery.

After completion of MR imaging, the presence or absence of the intra-coronary thrombus was confirmed using an interventional x-ray unit, which is considered to be the gold-standard. Immediately after x-ray angiography, MR myocardial late enhancement imaging was performed for visualization of the corresponding infarct areas.

Imaging of Coronary Stent Thrombosis

Coronary in-stent thrombosis was induced by x-ray guided placement of internally glue coated (thrombogenic) MR translucent stents. Five stents (3*LAD, 2*LCX) were placed in 3 female domestic swine. Following stent placement, intra-coronary delivery of the fibrin binding MR contrast agent (60 μmol) was performed into the left main coronary artery over ˜3 min followed by a saline flush (over ˜30 s). Similar to the Gd-DTPA labeled clot experiment, free-breathing bright blood coronary MR angiography and black blood MR thrombus imaging of the LAD or LCX were performed 1) before and after stent placement and 2) before and immediately after injection of the fibrin binding MR contrast agent.

After completion of MR imaging and follow up x-ray angiography, 2 in-stent thrombi were removed from the arteries and submitted for ICP analysis for determination of Gd concentration. No MR late enhancement images were acquired to guarantee an accurate [Gd] count of the contrast agent.

Imaging Protocols

Localization of Coronary Arteries

All scans were synchronized to the ECG with 3 electrodes placed on the mid-thorax and with imaging triggered on the R-wave to start in mid to late diastole. All scans were done during mechanically controlled free breathing using a commercial 5-element cardiac synergy receiver coil.

A non-triggered multislice (9 slices per stack) multistack (transverse, sagittal, coronal) steady state free precision (balanced FFE) scout scan (TR=2.5 ms, TE=1.9 ms, flip angel=55°, FOV=450 mm, matrix=128×128, in-plane resolution=3.5 mm, slice thickness=10 mm) was performed to localize the heart and the dome of the right hemidiaphragm. Subsequently, an ECG triggered and navigator-gated transverse 3D bTFE scan (TR=3.5 ms, TE=1.6 ms, flip angel=75°, FOV=400 mm, matrix=256×256, in-plane resolution =1.6*1.6 mm, slice thickness=3 mm) was performed to define the major axis of the left anterior descending (LAD) and left circumflex (LCX) coronary arteries.

Coronary MR Angiography

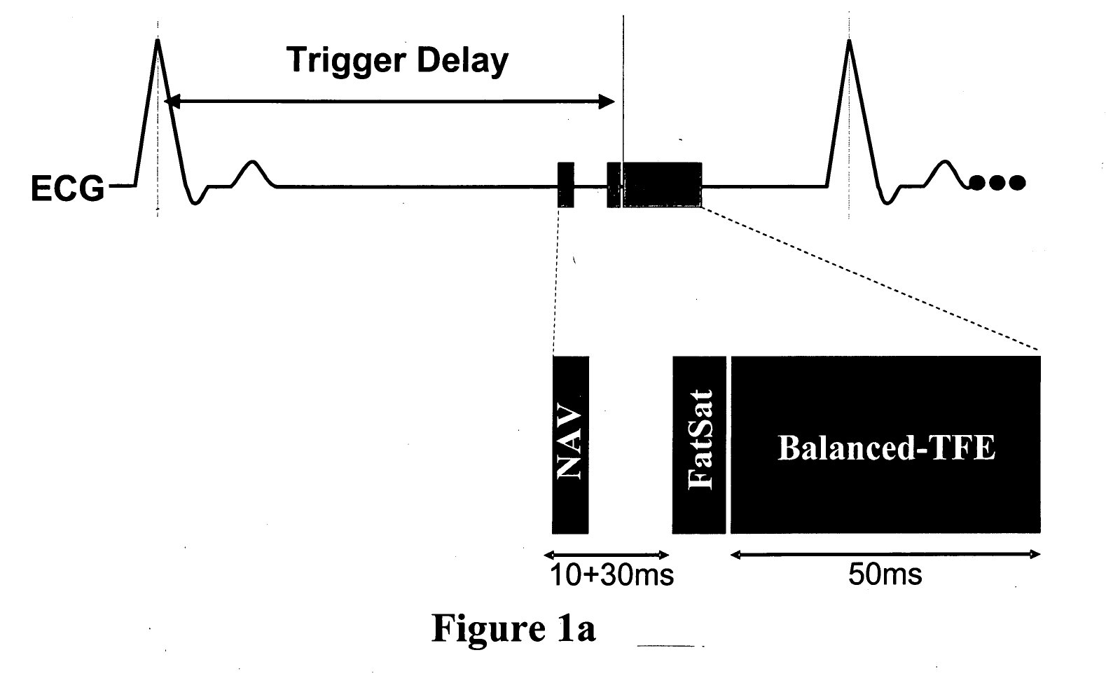

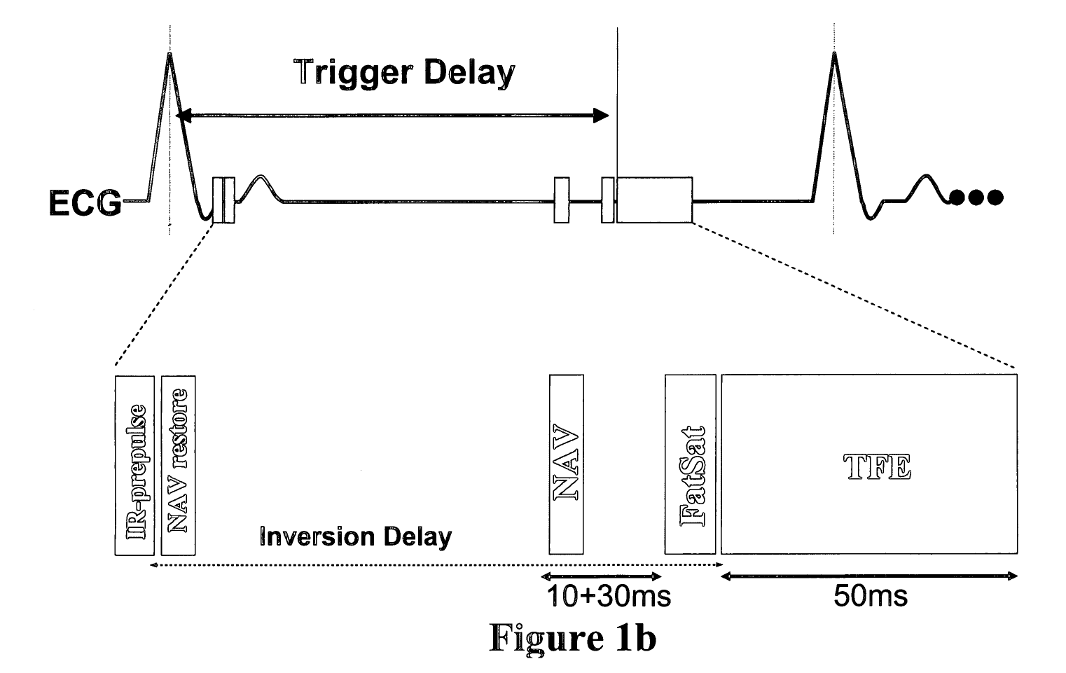

Using a 3-point planscan tool, the LAD and LCX were then imaged in double oblique planes using a magnetization prepared (T2prep) 3D bTFE coronary MRA sequence (FIG. 1a). Imaging parameters included FOV=320 mm, matrix=256*256, in-plane resolution=1.25*1.25 mm, slice thickness=3 mm, acquisition window=50 ms, TR/TE=5.4 ms/2.7 ms, flip angle=110°, start up cycles=20, and number of slices=12-15. Imaging time was ˜6-8 minutes. All imaging data were acquired in mid-diastole with the navigator placed on the dome of the right hemidiaphragm using a 5 mm gating window.

Coronary MR Thrombus Imaging

Thrombus imaging was performed in the same imaging plane as the high-resolution coronary MRA. Imaging parameters of the ECG triggered and navigator-gated T1 weighted black blood IR-TFE sequence (FIG. 1b) included FOV=320 mm, matrix=256*256, in-plane resolution=1.25*1.25 mm, slice thickness=3 mm, acquisition window=50 ms, TR/TE=4.7 ms/1.4 ms, partial echo, flip angle=30°, inversion time=285 ms (@ 90 bpm), and number of slices=12-15. Imaging time was ˜6-8 minutes. Similarly to the bright blood coronary MRA, mid-diastolic data acquisition was performed.

Myocardial MR Scar Imaging

Following Gd-labeled clot imaging and immediately after x-ray angiography, MR infarct imaging was performed after intra-venous administration of 1 mmol/kg Gd-DTPA. Seven short axis slices were acquired in subsequent breathholds using an ECG-triggered late enhancement technique. Imaging parameters include included FOV=320 mm, matrix=256*256, in-plane resolution=1.25*1.25 mm, slice thickness=10 mm, acquisition window =113 ms, TR/TE=7.5 ms/3.8 ms, partial echo, flip angle=15°, inversion time=250 ms. Data acquisition was performed in mid-diastole.

Signal-to-noise ratio (SNR) of thrombus was determined by manually segmenting the visually apparent thrombus area (in three adjacent slices) and calculating the mean signal (S). Noise (N) was determined within a region-of-interest (ROI) drawn outside of the animal. Contrast-to-noise ratio (CNR=(Sₜₕᵣₒₘᵤₛ−S_{blood/muscle})/N) was measured between thrombus and aortic blood (S_{blood}) and thrombus and adjacent muscle (S_{muscle}), respectively.

Results

All six animals completed both MR and x-ray angiographic imaging of the LAD and LCX. One animal died before completion of the myocardial scar examination. Three Gd-labeled fibrinogen clots and 5 MR-lucent stents were successfully delivered/placed under x-ray guidance in the left coronary system.

In-vitro Imaging of Gd-DTPA Labeled Fibrin Clots

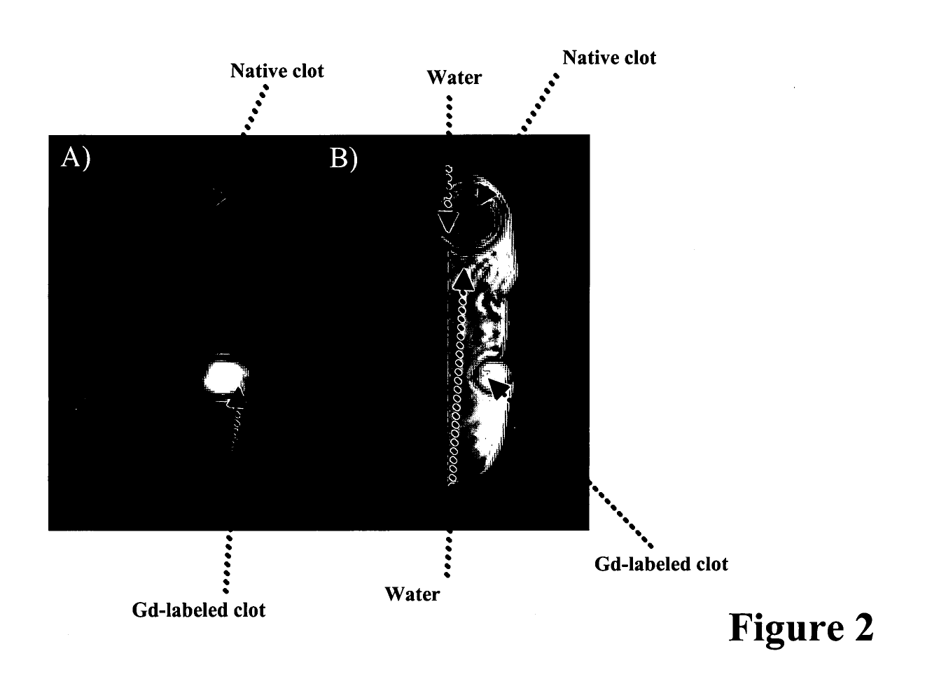

Gd-labeled fibrin clots appeared as bright spots on the otherwise hypo-intense IR-TFE images and had considerably higher CNR and SNR than native unlabeled clots (Gd-labeled clot: CNR<550; SNR<600 vs. native unlabeled clot: CNR<8SNR<18) (FIG. 2a). Both Gd-labeled clots and native clots had intermediate CNR and SNR on bTFE images (Gd-labeled clot: CNR<23;SNR<112 vs. native unlabeled clot: CNR<60;SNR<35), but were less well-delineated than Gd-labeled clots on IR-TFE images (FIG. 2b).

In-vivo Imaging of Gd-DTPA Labeled Fibrin Clots

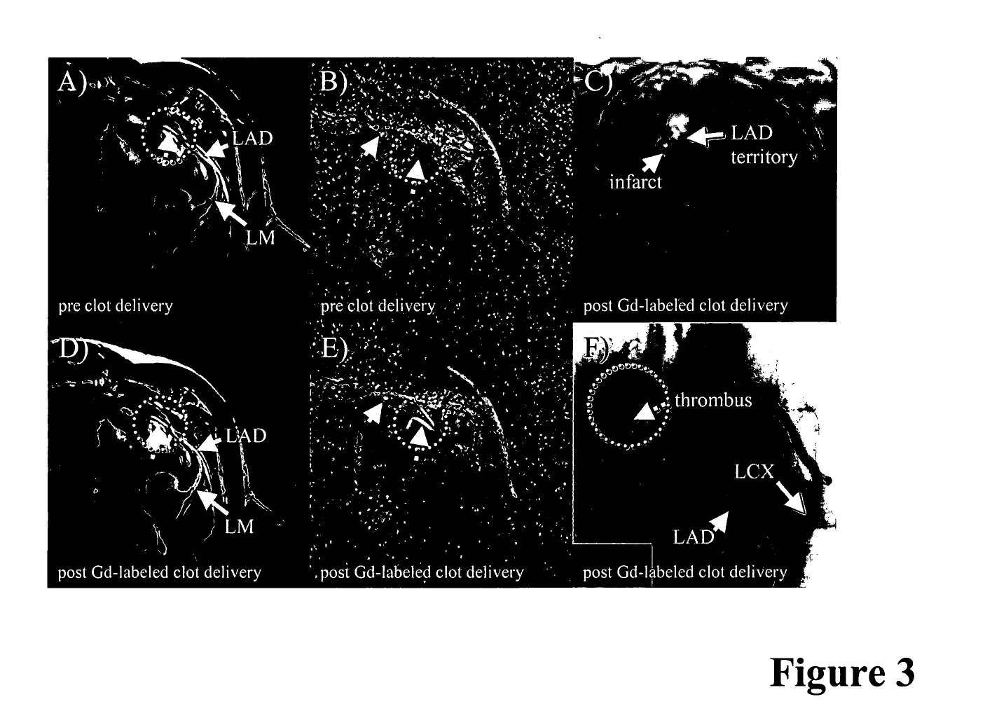

All three animals successfully completed both MR and x-ray angiographic imaging of the LAD and LCX. Five of the six Gd-labeled clots were clearly visible on the IR-TFE MR images (FIG. 3e) and were subsequently confirmed by x-ray angiography (FIG. 3f) and MR late enhancement imaging (for scar or infarct imaging) (FIG. 3c). One x-ray-confirmed clot was not visible on MR as it was outside of the imaging volume. Consistent with in vitro data (FIG. 2a), bright blood bTFE images (FIG. 3a, d) provided minimal information with respect to presence and location of the Gd-labeled fibrin clots. Average contrast-to-noise (CNR) values between Gd-DTPA labeled clots (˜250 μM Gd) and immediately surrounding tissues were 21±8 (SNR_{clot=}24±9) on the IR-TFE images (FIG. 3e).

In-vivo Imaging of Coronary Stent Thrombosis

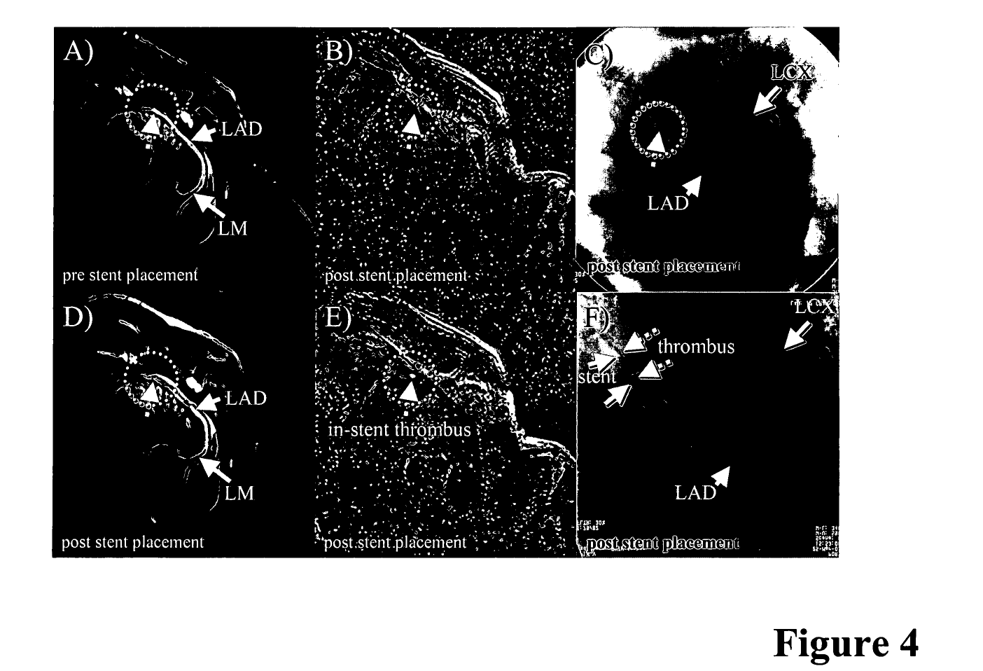

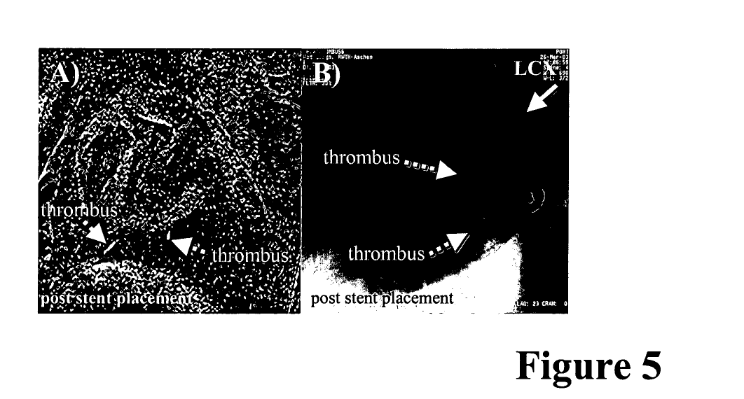

All five MR-lucent stents were successfully placed and in-stent thrombus was observed in all 5 stents after injection of the fibrin binding MR contrast agent (FIGS. 4, 5) with an average SNR and CNR of 11±2 and 9±2. Four of these clots were subsequently confirmed by x-ray angiography (FIGS. 4, 5). One of the MR detected clots was only visible on the first post contrast agent dataset but was absent on subsequent IR-TFE scans. Consistent with this finding, no clot was seen on the subsequent x-ray angiogram (Table 2). Similar to the Gd-labeled fibrin clot experiment, bright blood coronary MRA provided minimal information with respect to presence and location of in-stent thrombus. Chemical analysis of two thrombi resulted in 99 μM and 147 μM Gd, consistent with Gd concentrations expected from in vitro experiments.

As expected from in vitro studies, only a relatively small amount of the fibrin binding contrast agent (˜25 mM) was required (corresponding to 100 mM Gadolinium) for ready detection of intra-stent thrombus. Intra-coronary delivery of the contrast agent (60 μmol) over a ˜3 minute period was sufficient for this fibrin-targeted agent to bind to intra-coronary fibrin clots and to create a high enough signal for immediate detection of intra-coronary thrombus. No contrast uptake was observed in surrounding tissues either in coronary or in ventricular blood.

MR Lucent Stents

All stents were successfully placed in the coronary arteries under x-ray guidance and all glue coated (thrombogenic) stents provoked local thrombosis as demonstrated on MRI and subsequently confirmed by x-ray angiography. In addition, no stent related artifacts, typically due to local field inhomogeneities or local RF attenuation, were observed in any of the animals.

Coronary MRA and Thrombus Imaging

The use of a flow independent black blood inversion recovery sequence together with a T1 shortening contrast agent allowed for imaging of Gd-labeled fibrin clots and coronary stent thrombosis with excellent delineation of clot/thrombus from surrounding myocardium and blood. In contrast, bright blood coronary MRA provided only minimal information with respect to the presence and location of intra-coronary thrombus. The combination of triggered, mid-diastolic image acquisition together with navigator-based respiratory motion compensation provided artifact-free visualization of intra-stent thrombus. Furthermore, although a relatively course spatial resolution of 1.25×1.25×3 mm was used, good depiction of in-stent thrombosis was achieved.

### Example 2

### Coronary MR Angiography and Coronary Thrombus Imaging (Cardiac Triggering and Navigator Gating)—Systemic Delivery of Contrast Agent

The experiment sought to test the feasibility of direct MR imaging of acute coronary thrombosis using systemic injection of a fibrin-binding contrast agent in an in vivo swine model of coronary thrombosis. Free-breathing coronary MR angiography and thrombus imaging were performed on three female domestic swine (50 kg) in the supine position using a 1.5 T Philips Gyroscan Intera short-bore MRI scanner (Philips Medical Systems, Best, NL). The MRI system was equipped with MASTER gradients (23 mT/m, 105 mT/m/ms), an advanced cardiac software patch (R9.1.1), and a 5-element cardiac synergy receiver coil.

Animal Protocol

After intramusculary premedication with 0.5 ml atropine and 0.2 ml azaperone/kg body weight, an aqueous solution of pentobarbital (1:3) was administered intravenously through one of the ear veins. The animals were intubated and mechanical ventilation was maintained throughout the entire experiment. A 9F sheath (Cordis, Roden, NL) was placed surgically in the right carotid artery.

Thrombus Preparation and Delivery

Human fibrinogen, human thrombin (10 NIH U), 25 mM CaCl2 and blood were mixed in a syringe and allowed to incubate for 30 minutes at room temperature. After incubation, any remaining supernatant was removed.

Five thrombi were delivered under x-ray guidance to the right coronary artery (RCA), left anterior descending (3×LAD), and left circumflex (LCX) of the three swine (50 kg, F). Subsequently, free-breathing bright blood steady state free precession (SSFP) (=coronary MRA; FIG. 1(C)) and black blood inversion-recovery (IR) TFE (=MR thrombus imaging; FIG. 1 (d)) 3D coronary artery imaging of the RCA, LAD or LCX were performed before and after systemic injection of the fibrin binding contrast agent set forth in Example 1 (7.5 μmol/kg). MRI was repeated until 2 hours post injection. After completion of MR imaging, coronary thrombosis was confirmed by x-ray angiography and autopsy.

Localization of Cardiac Landmarks and Coronary Arteries

All scans were performed using a commercial 5-element cardiac synergy receiver coil (Philips Medical Systems, Best, NL). A non-ECG-triggered multislice (9 slices per stack) multistack (transverse, sagittal, coronal) steady state free precision (balanced FFE) scout scan (repetition time (TR)=2.5 ms, echo time (TE)=1.9 ms, flip angel=55°, field-of-view (FOV)=450 mm, matrix=128×128, in-plane resolution=3.5 mm, slice thickness=10 mm) was performed to localize the heart and the dome of the right hemidiaphragm. Subsequently, an ECG-triggered and navigator-gated transverse 3D bTFE scan (TR=3.5 ms, TE=1.6 ms, flip angle=75°, FOV=400 mm, matrix=256×256, in-plane resolution=1.6×1.6 mm, slice thickness=3 mm) was performed to define the major axes of the LAD and LCX coronary arteries.

Coronary MR Angiography

Using a 3-point planscan tool, the LAD and LCX were imaged in double oblique planes using a previously described magnetization prepared (T2prep) 3D bTFE coronary MRA sequence. Imaging parameters include FOV=320 mm, matrix=256×256, in-plane resolution=1.25×1.25 mm, slice thickness=3 mm, acquisition window=50 ms, TR/TE=5.4 ms/2.7 ms, flip angle=110°, start up cycles=5, and number of slices=12-15. Imaging time was 5-8 minutes. All data were acquired in mid-diastole (acquisition window =50 ms) with the navigator placed on the dome of the right hemidiaphragm using a 5 mm gating window.

In-Vivo Coronary MR Thrombus Imaging

In-vivo thrombus imaging was performed in the same imaging plane as that used for the coronary MRA. Imaging parameters of the ECG triggered and navigator gated T1 weighted black blood IR-TFE sequence include FOV=320 mm, matrix=256×256, in-plane resolution=1.25×1.25 mm, slice thickness=3 mm, acquisition window=50 ms, TR/TE=4.7 ms/1.4 ms, partial echo, flip angle=30°, inversion time=285 ms @90 bpm), and number of slices=12-15. Imaging time was ˜6-8 minutes. As for the bright blood coronary MRA, mid-diastolic data acquisition (acquisition window =50 ms) was performed with a right hemidiaphragmatic navigator using a 5 mm gating window.

Results

90 minutes after contrast injection, all thrombi (RCA, LAD, LCX) were visible on T1-weighted IR MR images. The presence and location of coronary thrombus was confirmed by MDCT, x-ray angiography, and autopsy. Analysis of excised thrombi by mass spectrometry confirmed the expected Gd concentration.

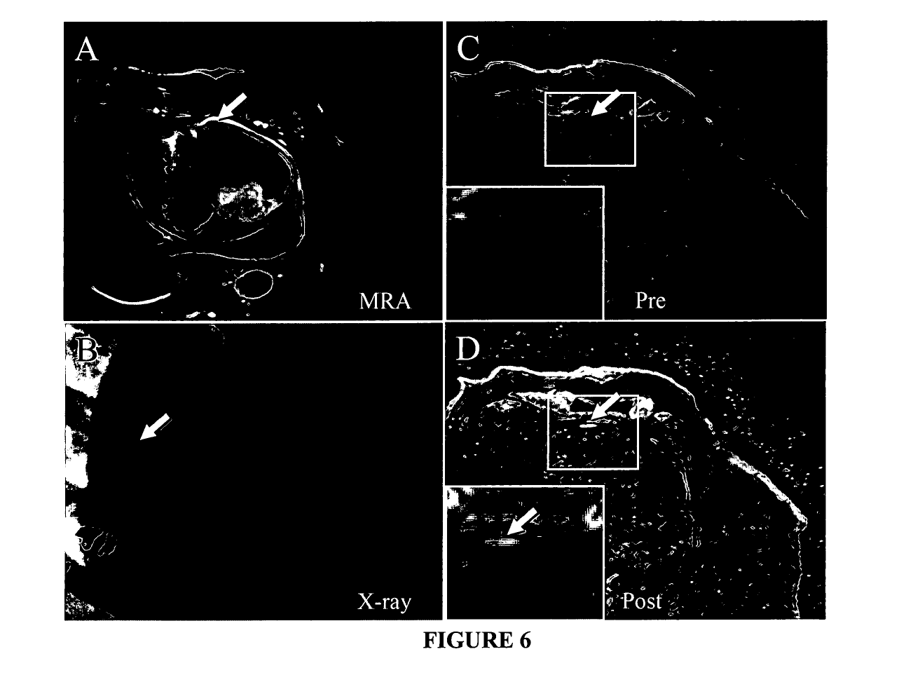

FIG. 6 demonstrates the in vivo MR imaging of coronary thrombosis with systemic injection of the fibrin binding contrast agent described in Example 1. Bright blood bTFE (A) and black blood IR-TFE images before (C) and after (D) systemic injection of the fibrin-binding MR contrast agent are shown. Good thrombus depiction (arrow) is evident in the post-contrast image (D). The thrombus was subsequently confirmed (arrow) by x-ray angiography (B); to allow comparison with MR images, the orientation of the X-ray image was horizontally reversed.

The experiment successfully demonstrated the feasibility of in vivo MR imaging of acute coronary thrombosis using a fibrin-targeted contrast agent and systemic contrast agent injection in the presence of respiratory and cardiac motion. Applications include detection of acute coronary syndromes, atrial clots, and suspected pulmonary embolism.

### Example 3

### Coronary MR Angiography Coronary Thrombus Imaging and Pulmonary Embolism Imaging Using Cardiac Triggering and Navigator Gating—Systemic Delivery of Contrast Agent

The differential diagnosis of acute chest pain is challenging, particularly in patients with normal ECG, and may include coronary thrombosis and/or pulmonary emboli. The aim of this study was the investigation of a fibrin-specific contrast agent (as described in Example 1) for molecular targeted imaging of coronary thrombosis and pulmonary emboli.

Animal Protocol

Coronary thrombus and pulmonary embolus MR imaging were performed on 7 healthy swine (48-52 kg BW). After premedication with 0.5 ml IM atropine, 0.2 ml IM azaperone/kg bodyweight, and 0.1 ml ketamine/kg bodyweight, an aqueous solution of pentobarbital (1:3) was administered intravenously via an ear vein as needed. The animals were intubated and mechanical ventilation was maintained throughout the entire experiment. A 9F sheath (Cordis, Roden, NL) was placed surgically in the right carotid artery and a 1 6F sheath (Cordis, Roden, NL) was placed in right iliac vein.

Fresh clots from human blood were engineered ex vivo as described previously and delivered in the iliac vein and coronary arteries of seven swine under x-ray guidance. For pulmonary embolism, five to seven thrombi per swine were dragged into a 12 F sheath and then delivered via the 16 F sheath in the iliac vein by washing the sheath with saline. Coronary thrombi were delivered via a 9F guiding catheter into the LAD (n=3), RCA (n=1) and LCX (n=1) under x-ray guidance. As a control in a further pig, pulmonary emboli were delivered and imaged without application of any extrinsic contrast medium. In another pig, standard extracellular contrast was given at clinical dose (0.1 mmol/kg BW Gd-DTPA, Magnevist™, Schering, Berlin, Germany). All pigs were heparinized to avoid additional clotting.

After clot delivery, the pigs were transferred to the MR unit. All MR studies were carried out on a 1.5T Gyroscan Intera whole body MR system (Philips Medical Systems, Best, NL, 23 mT/m, 219 μs rise time). A four element body wrap around Synergy coil was used for signal reception. All subjects were examined in the supine position. Identical molecular MR imaging sequences of the lungs (coronal slice orientation) and the coronary arteries (double oblique slice orientation) were performed prior to contrast media administration and repeated for 2h after systemic delivery of 0.0075 mmol of the fibrin binding contrast agent in Example 1/kg BW via an ear vein. MR imaging included a navigator-gated free-breathing cardiac triggered 3D inversion-recovery black-blood gradient-echo sequence and a spoiled breath-hold gradient-echo sequence. MR images were analyzed by two investigators and contrast-to-noise ratio (CNR) between the thrombus and the blood pool were assessed. Subsequently, 16 row multislice CT was performed for comparison. Finally, the animals were sacrificed and the clots were removed from the pulmonary vascular bed for the assessment of Gd-concentration in the clots.

Pulmonary MR Imaging Sequences

MR imaging of the lungs consisted of a navigator-gated free-breathing cardiac triggered inversion recovery and fat suppressed 3D black-blood gradient echo sequence (TR 4.0 ms, TE 1.3 ms, flip angle 30°, filed-of-view 400×400 mm, 256×256 matrix reconstructed with a 512×512 matrix to a 1.5×1.5×2 mm voxel size including zero filling in z-direction). For enhanced contrast between the thrombus and the surrounding blood pool, heart rate specific inversion times were used maintaining complete black-blood properties in gradient echo imaging. 3 6 excitations per R-R interval resulted in a 145 ms acquisition window. Data acquisition was timed to late diastole and central k-space data were acquired first in order to minimize potential motion artifacts. For lung imaging 80 two mm thick coronal slices were acquired.

Coronary MR Imaging Sequences

For coronary MR imaging, a transverse steady-state free-precession scout scan was first performed for planning of the subsequent targeted double-oblique coronary MR imaging scans. Targeted coronary MR imaging included a navigator-gated free-breathing cardiac triggered T2-prepared 3D steady-state free precession coronary bright-blood coronary MR angiography sequence for visualization of the anatomy of the coronary artery lumen. MR thrombus imaging was performed similarly to lung imaging by use of a navigator-gated free-breathing cardiac triggered inversion recovery and fat suppressed 3D black-blood gradient echo sequence. Spatial resolution of the coronary scan was increased by reducing the field-of-view to 320 mm. The resultant reconstructed spatial resolution was 0.6×0.6×1.5 mm including zero filling in z-direction (TR was 4.4 ms and TE was 1.4 ms, flip angle was 30°). 12 excitations per R-R interval resulted in a more brief, 56 ms end-diastolic acquisition window, allowing for further reduction of intrinsic cardiac motion artifacts.

For coronary bright-blood MR-angiography as well as for coronary black blood thrombus imaging, 24 1.5 mm thick slices were acquired with the imaging plane adjusted to the main axis of the coronary artery using a three-point planscan tool.

2D Selective Navigator

For free-breathing data acquisition, all sequences were equipped with a right hemi-diaphragmatic prospective real-time navigator for respiratory motion artifact suppression. A gating window of 5 mm was used. As the inversion pulse in the inversion recovery black blood sequences may reduce navigator performance, the excitation angle of the navigator beam was increased to 45 degrees and the navigator diameter was set to 50 mm. This allowed for high navigator performance with navigator efficiency always higher the 50%. The navigator restore pulse was switched off, as this pulse may result in a ‘spin labeling’ of the pulmonary blood, resulting in reduced black blood properties.

Pulmonary MDCT-Angiography

Multislice CT was performed for comparison because it can detect pulmonary embolism in a swine model and is currently used clinically in patients with suspected pulmonary embolism. CT scanning of the lung was performed with 16×0.75 mm collimation (Somatom Sensation, Siemens, Erlangen, Germany) 120 kV tube voltage, 300 mm reconstruction field-of-view, 15 mm table feed per rotation after bolus application of 90 ml non-ionic contrast material (Ultravist 370, Schering, Berlin, Germany) at a flow-rate of 3.5 ml/sec. Axial images with 2 mm reconstruction increment and coronal MPRs from 1.0/0.6 mm reconstructions were used.

Results

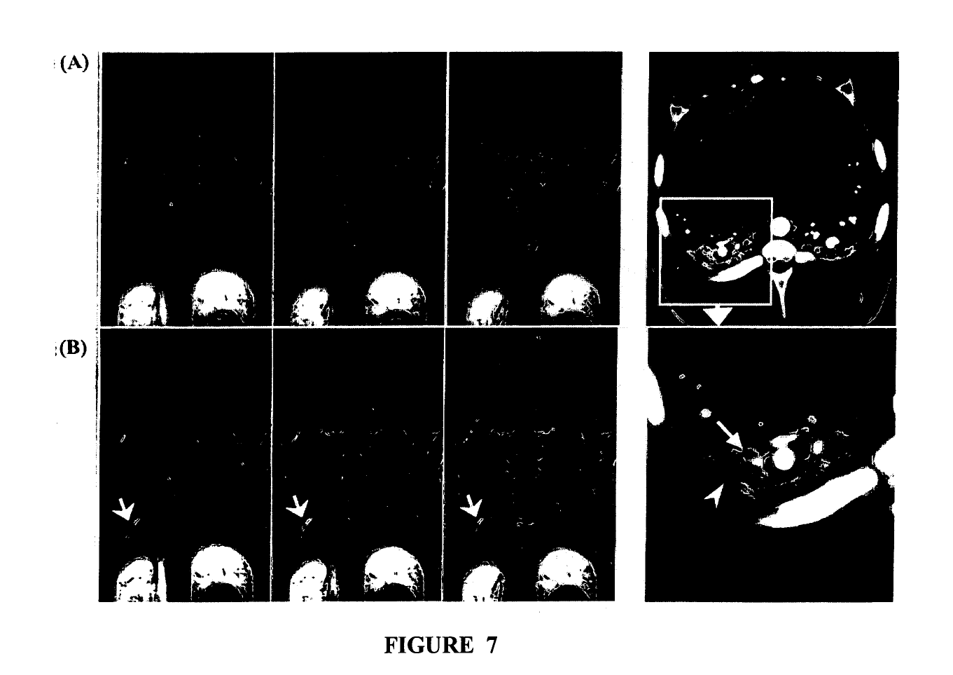

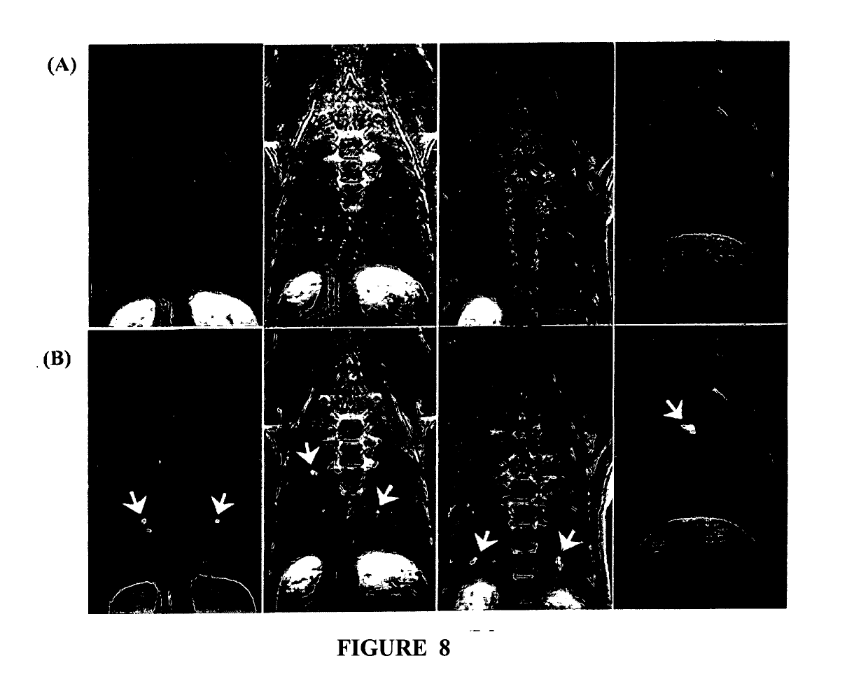

Prior to contrast media administration, all thrombi were not visible in the pulmonary vessels nor in the coronary arteries. After contrast media administration, numerous pulmonary emboli, three emboli in the right heart, and five coronary thrombi were selectively visualized with a bright signal on MR images, while the surrounding tissue and the blood pool were signal suppressed. A high gadolinium concentration in the thrombi was found resulting in a high CNR on MR images. All thrombi were proven by x-ray, Multislice-CT, or macroscopically. The fibrin binding contrast agent thus allows for selective molecular imaging of fresh coronary, cardiac, and pulmonary clots. See FIGS. 7 and 8.

A number of embodiments of the invention have been described. Nevertheless, it will be understood that various modifications may be made without departing from the spirit and scope of the invention. Accordingly, other embodiments are within the scope of the following claims.

1. A method for determining the presence or absence of a stationary target in a bodily location of an animal, said bodily location subject to physiologic motion, said method comprising:
a) administering a MRI contrast agent to said animal, said MRI contrast agent capable of binding to said stationary target;
b) allowing said MRI contrast agent to bind to said stationary target; and
c) acquiring one or more MR images of said bodily location, wherein said acquisition of said one or more MR images is capable of reducing motion artifacts in said one or more MR images.

2. The method of claim 1, wherein said physiologic motion is periodic motion.

3. The method of claim 2, wherein said periodic motion is due to respiratory motion or cardiac motion of said animal.

4. The method of claim 1, wherein said physiologic motion is due to musculoskeletal motion of said animal.

5. The method of claim 3, wherein said physiologic motion is due to both respiratory and cardiac motion of said animal.

6. The method of claim 1, wherein said reduction of motion artifacts is achieved by acquiring MR data at a predetermined time during said animal's cardiac cycle.

7. The method of claim 1, wherein said reduction of motion artifacts is achieved by acquiring MR data during a predetermined period of said animal's respiratory cycle.

8. The method of claim 6, wherein said MR data acquisition at a predetermined time during said animal's cardiac cycle occurs by coordinating said MR data acquisition with a physiologic electrical or pressure signal of said animal.

9. The method of claim 8, wherein said physiologic electrical or pressure signal is selected from the group consisting of an ECG signal, a heartbeat, and a pulse of said animal.

10. The method of claim 8, wherein said pressure signal of said animal is detected using an acoustic technique, an ultrasound technique, or a transducer.

11. The method of claim 9, wherein said physiologic signal is an ECG signal, and wherein said MR data acquisition occurs during mid- or late-diastole of said ECG signal.

12. The method of claim 7, wherein said acquisition of MR data during a predetermined period of said animal's respiratory cycle occurs by coordinating said MR data acquisition with a location of said animal's diaphragm, liver, or lung.

13. The method of claim 12, wherein said location of said diaphragm, liver, or lung is determined using a MR navigator, a tracking MR navigator, high speed MR projection images, or full MR images.

14. The method of claim 7, wherein said predetermined period of said respiratory cycle is determined by using a respiratory bellows.

15. The method of claim 7, wherein said predetermined period of said animal's respiratory cycle is the end of expiration.

16. The method of claim 7, wherein said predetermined period of said animal's respiratory cycle is a breath-hold of said animal.

17. The method of claim 1, wherein said one or more MR images are acquired using a contrast-enhancing imaging pulse sequence.

18. The method of claim 17, wherein said contrast-enhancing imaging pulse sequence is capable of suppressing the MR signal of in-flowing blood and is further capable of enhancing the MR signal of said stationary target.

19. The method of claim 17, wherein said contrast-enhancing imaging pulse sequence comprises a turbo field echo sequence, a spoiled gradient echo sequence, or a high speed 3D acquisition sequence.

20. The method of claim 17, wherein said contrast-enhancing imaging pulse sequence comprises a black blood MR angiography sequence.

21. The method according to claim 20, wherein said black blood MR angiography sequence comprises a fast spin echo sequence, a flow-spoiled gradient echo sequence, an inversion recovery sequence, a double inversion recovery sequence, a fast gradient echo sequence, or an out-of-volume in-flow suppression sequence.

22. The method of claim 1, wherein said stationary target comprises a protein.

23. The method of claim 22, wherein said protein is selected from the group consisting of fibrin, collagen, elastin, decorin, and a Toll-like receptor.

24. The method of claim 1, wherein said stationary target is selected from the group consisting of oxidized LDL, matrix metalloproteinases, LTB4, and hyaluronan.

25. The method according to claim 1, wherein said stationary target is selected from the group consisting of a thromboembolism, an aneurism, an embolism, a thrombus, a tumor, a region of fibrosis, a region of infarcted tissue, a region of ischemic tissue, an atherosclerotic plaque, and a vulnerable plaque.

26. The method of claim 1, wherein said stationary target is a region of heart, liver, kidney, or lung tissue.

27. The method of claim 26, wherein said heart, liver, kidney, or lung tissue is ischemic or infarcted.

28. The method of claim 17, wherein said contrast-enhancing imaging pulse sequence comprises an in-flow-independent technique, said in-flow-independent technique capable of enhancing the contrast ratio of a magnetic resonance signal of said stationary target having said MRI contrast agent bound thereto relative to a magnetic resonance signal of background blood or tissue.

29. The method of claim 28, wherein said background blood is in-flowing blood.

30. The method of claim 28, wherein said background tissue is fat, muscle, or tissue.

31. The method of claim 28, wherein said in-flow-independent technique comprises an inversion-recovery prepared sequence, a saturation-recovery prepared sequence, a T₂ preparation sequence, or a magnetization transfer preparation sequence.

32. The method of claim 1, wherein said bodily location is the heart, lung, kidneys, great blood vessels, or the liver of said animal.

33. The method of claim 32, wherein said bodily location is the myocardium, an atrium, a ventricle, a coronary artery, or a valve of the heart.

34. The method of claim 1, wherein said bodily location is a skeletal joint.

35. The method of claim 1, wherein said contrast agent is selected from the group consisting of:

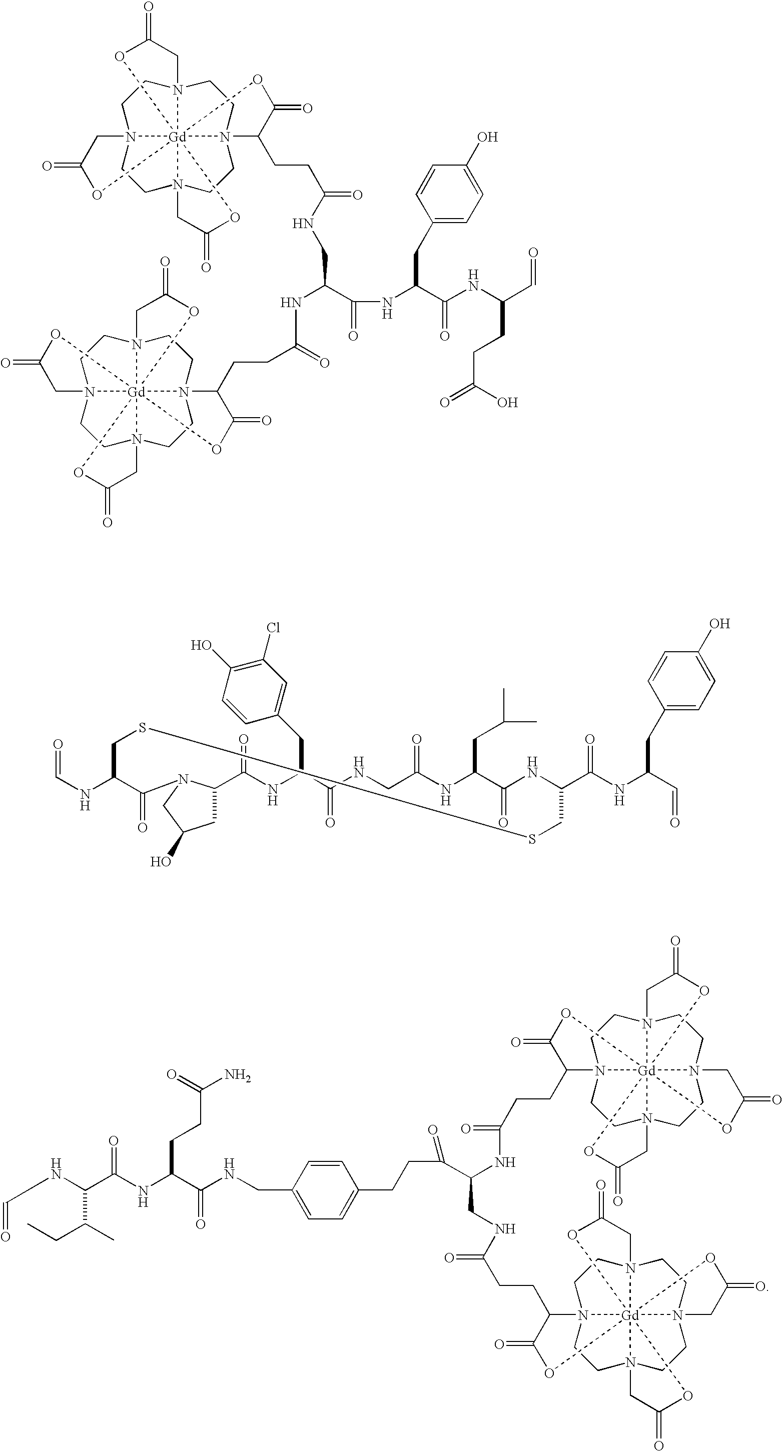

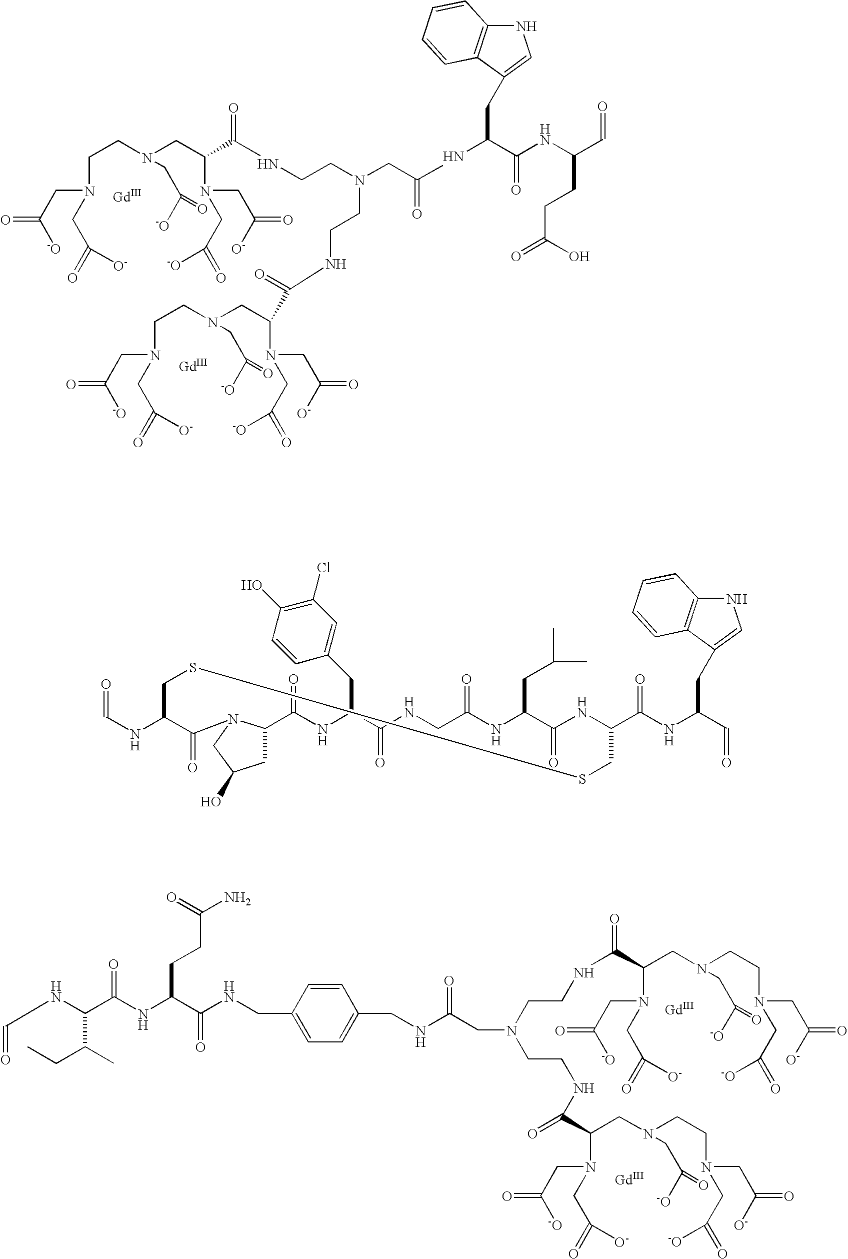

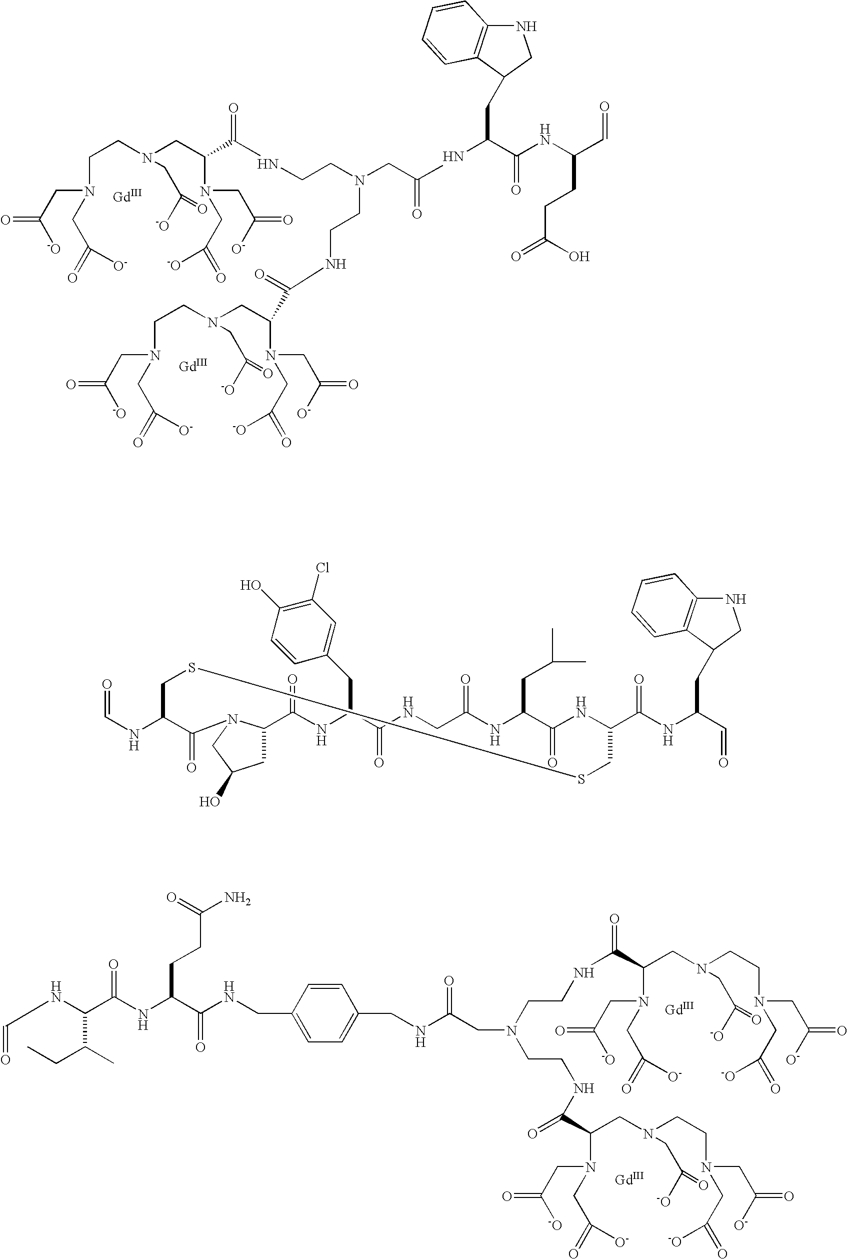

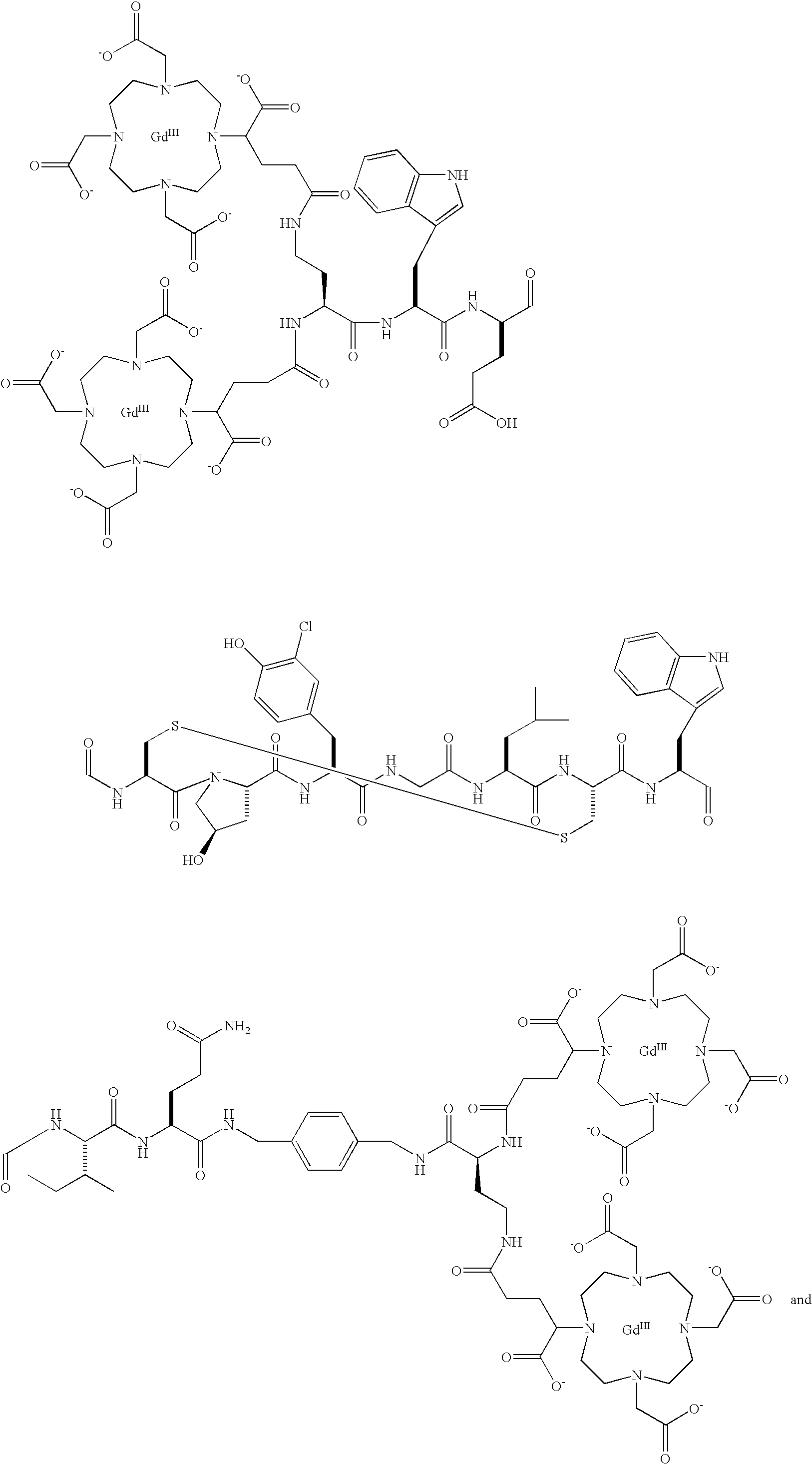

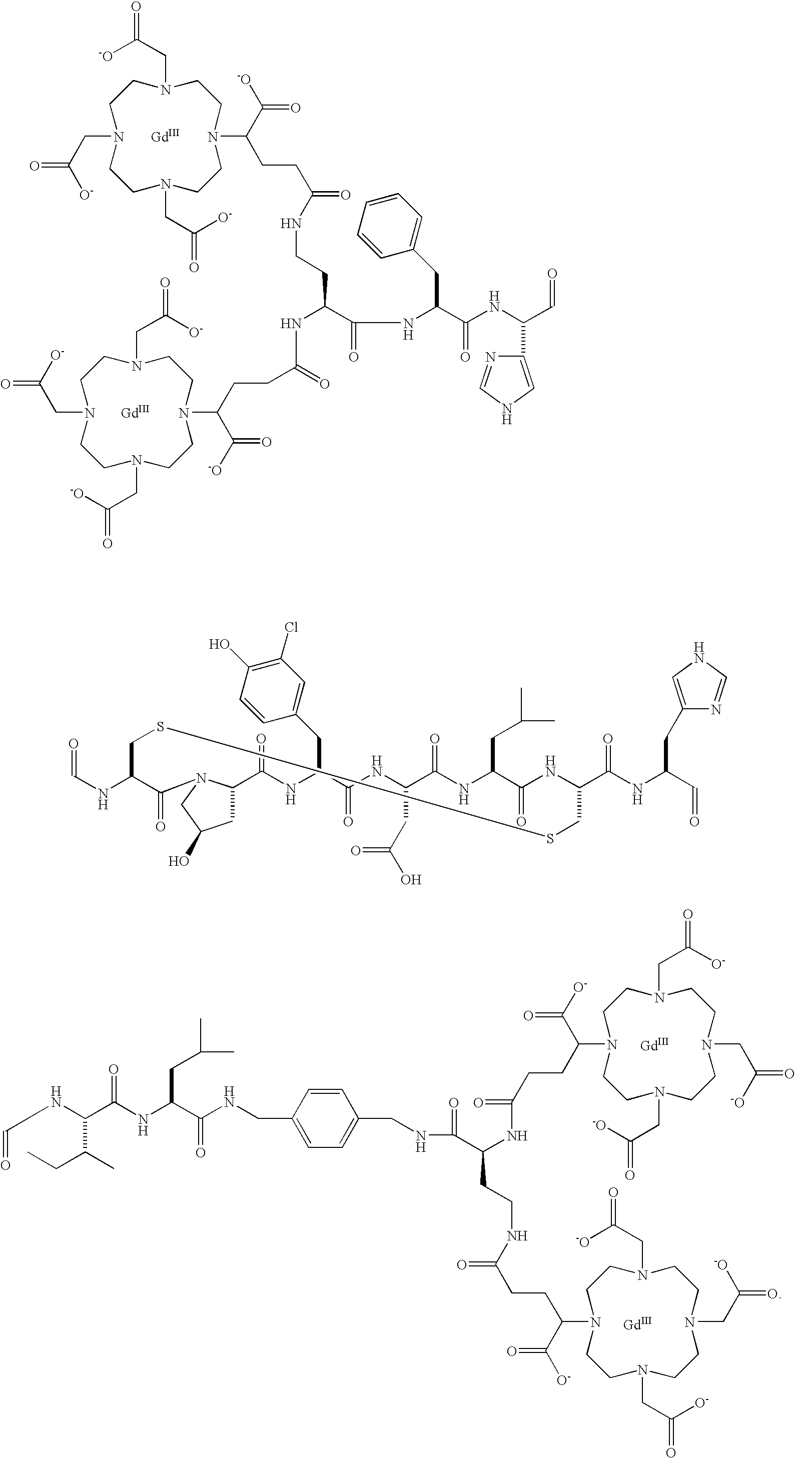

36. The method of claim 1, wherein said animal is a human.

37. A method for determining the presence or absence of a stationary target in a bodily location of an animal, said bodily location subject to physiologic motion, said method comprising:
a) administering a MRI contrast agent to said animal, said MRI contrast agent capable of binding to said stationary target;
b) allowing said MRI contrast agent to bind to said stationary target;
c) acquiring one or more MR images of said bodily location, said acquisition of said one or more MR images capable of reducing motion artifacts in said one or more MR images; and
d) examining said one or more MR images, wherein said stationary target is determined to be present when a contrast-enhanced region is observed.

38. The method of claim 37, wherein said presence of said stationary target is correlated with a pathology of said animal.

39. The method of claim 38, wherein said pathology is selected from the group consisting of a coronary syndrome, a coronary stent thrombosis, fibrosis of the lung, ischemic myocardial tissue, infarcted myocardial tissue, a pulmonary embolism, and a deep venous thrombosis.
